(19) United States
(12) Patent Application Publication
(10) Pub. No.: US 2016/0213791 A1
(43) Pub. Date: Jul. 28, 2016

(54) METHODS AND SENSORS FOR DETECTING A BIOLOGICAL PARAMETER

(71) Applicant: PREGTECH PTY LTD, New South Wales (AU)

(72) Inventor: Nicolas Hans VOELKER, Blackwood (AU)

(73) Assignee: PREGTECH PTY LTD, New South Wales (AU)

(21) Appl. No.: 14/913,733

(22) PCT Filed: Aug. 22, 2014
(86) PCT No.: PCT/AU2014/000835

§ 371 Date: Feb. 23, 2016

(30) Foreign Application Priority Data
Aug. 23, 2013 (AU) 2013903197

(51) Int. Cl. A61K 49/00

(52) U.S. Cl. CPC A61K 49/0006; A61K 49/0004

(57) ABSTRACT

The present disclosure relates to methods for detecting a biological parameter and to sensors for detecting a biological parameter. Certain embodiments of the present disclosure provide a method of detecting a biological parameter in a subject, the method comprising: implanting a sensor into the subject, wherein the sensor comprises an optical reflectance property between 400 and 1200 nm which is responsive to the biological parameter; detecting an optical reflectance property between 400 and 1200 nm through the skin of the subject from the implanted sensor, and using the optical reflectance property to detect the biological parameter.

### PRIORITY CLAIM

This application claims priority to Australian provisional patent application number 2013903197 filed on 23 Aug. 2013, the contents of which are hereby incorporated by reference.

### FIELD

The present disclosure relates to methods for detecting a biological parameter and to sensors for detecting a biological parameter.

### BACKGROUND

Advances in biology and material sciences have opened the possibility for using sensors to detect various parameters of biological relevance. Such sensors are typically referred to as “biosensors”. These biosensors hold the promise that they can be used to provide an improved assessment of various biological parameters, with a consequential improvement in the management of the health and biology of humans and animals.

However, such biosensors often suffer from one or more disadvantages. For example, the type of signal output from a biosensor may not compatible with the detection of a biological parameter in a biological setting and/or there may be issues with the detection of the signal output in a biological setting. These issues are often due in large part to the fact that such biosensors operate in a complex biological milieu, which may interfere with detection of the relevant biological parameter and/or interfere with the signal to be detected.

These issues are particularly relevant for implantable biosensors. Not only must the sensor operate in a complex biological setting, but the surrounding tissue may also provide a barrier to reliable detection of the signal. For example, even in the case where a biosensor is implanted under the skin, the detection of signals from the biosensor still remains problematic.

Other important considerations with respect to implantable biosensors are the biocompatability of the biosensor and the issue of whether breakdown of the sensor may result in the generation of undesirable and/or toxic breakdown products. Such considerations are becoming increasingly important for biosensors that are to be implanted for a significant amount of time and/or for sensors that are to be implanted into animals of agricultural relevance. In the latter case, there is also an issue as to whether products arising from the implantation and/or degradation of the biosensor into animals may potentially compromise any products for human consumption.

For example, there is a need for biosensors that can be used to detect biologically important molecules in agricultural animals, such as hormones indicative of ovulation status or hormones indicative of pregnancy. Biosensors could be used, for example, to detect such hormones in the milk of the animals, or could be implanted into the animals to allow detection of the hormones, at a desired time. In this way, it would be possible to determine whether an animal is suitable for insemination or whether the animal is already pregnant, thereby providing significant economic benefits to animal management.

In humans there are a variety of situations where biosensors could be used to provide a read-out of a biological parameter indicative of a particular disease, condition or state. For example, biosensors could also be used to determine ovulation status or pregnancy in female humans. Such parameters could be detected in vivo, or could be detected for example in biological fluids.

Accordingly, the development of biosensors that can be used to detect biological parameters with improved performance would therefore be advantageous.

### SUMMARY

The present disclosure relates to methods for detecting a biological parameter and to sensors for detecting a biological parameter.

Certain embodiments of the present disclosure provide a method of detecting a biological parameter in a subject, the method comprising:
implanting a sensor into the subject, wherein the sensor comprises an optical reflectance property between 400 and 1200 nm which is responsive to the biological parameter;
detecting an optical reflectance property between 400 and 1200 nm through the skin of the subject from the implanted sensor, and
using the optical reflectance property to detect the biological parameter.

Certain embodiments of the present disclosure provide a method of determining the ovulation status of a subject, the method comprising:
implanting into the subject a sensor comprising one or more porous silicon layers and a luteinizing hormone binding molecule, wherein the sensor comprises an optical reflectance property between 400 and 1200 nm which is responsive to binding of leutinizing hormone to the sensor;
detecting an optical reflectance property between 400 and 1200 nm through the skin of the subject from the implanted sensor; and
determining the ovulation status of the subject on the basis of the optical reflectance property detected.

Certain embodiments of the present disclosure provide a method of determining the pregnancy status of a subject, the method comprising:
implanting into the subject a sensor comprising one or more porous silicon layers and a molecule that binds to an analyte indicative of pregnancy status, wherein the sensor comprises an optical reflectance property between 400 and 1200 nm which is responsive to binding of the analyte to the sensor;
detecting an optical reflectance property between 400 and 1200 nm through the skin of the subject from the implanted sensor; and
determining the pregnancy status of the subject on the basis of the optical reflectance property detected.

Certain embodiments of the present disclosure provide a sensor for detecting a biological parameter, the sensor comprising one or more porous silicon layers, wherein the sensor comprises an optical reflectance property between 400 and 1200 nm that is responsive to the biological parameter.

Certain embodiments of the present disclosure provide a method of detecting a biological parameter, the method comprising using a sensor as described herein to detect the biological parameter.

Certain embodiments of the present disclosure provide a method of determining the ovulation status of a subject, the method comprising implanting a sensor as described herein into a subject and using the implanted sensor to determine the ovulation status of the subject.

Certain embodiments of the present disclosure provide a method of determining the pregnancy status of a subject, the method comprising implanting a sensor as described herein into a subject and using the implanted sensor to determine the pregnancy status of the subject.

Certain embodiments of the present disclosure provide a method of identifying a subject, the method comprising using a sensor as described herein to tag the subject and thereby identify the subject.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows a scheme of the experimental setup for the reflection measurements of pSi reflectors through animal cadaver skin

FIG. 2 shows evolution of reflection spectra for rabbit skin (a, c, e, g) and guinea pig skin (b, d, f, h) skins in glycerol (a-f) and sucrose (g, h) solution. (a, b) single layer, (c, d) pSi microcavity, (e, f) pSi rugate filter, and (g, h) pSi single layer over a period of 24 h.

FIG. 3 shows photographs of pSi reflectors covered with skin samples. (a) Guinea pig skin onto pSi sample before incubation (zero time) in glycerol. (b) Exemplar pSi samples in glycerol solution. (c-d) Skin samples on pSi reflectors after 24 h incubation in glycerol. (e) Exemplar pSi reflectors in sucrose solution. (f) Skins onto pSi reflectors of (e) after 24 h treatment in sucrose. (g) pSi reflectors covered with skin samples after 24 h of treatment with glycerol. (h) Same samples as in (g), cleaned and incubated for 30 min in PBS.

FIG. 4 shows reflection spectra of pSi microcavities with the microcavity resonances centered at four different wavelengths in air and immersed in glycerol. The corresponding Q factor is depicted in each graph, for the spectra of pSi measured in glycerol solution (the horizontal axis changes in each figure, but the spectral window size was kept constant for comparison).

FIG. 5 shows evolution of reflection spectra of guinea pig skin in glycerol solution, using four pSi microcavities. Over each spectrum the time line of the experiment can be seen. The quality factor Q is presented in each plot.

FIG. 6A shows the white light reflectivity spectrum of porous silicon rugate film in aqueous medium. FIGS. 6B and 6C show SEM images of porous silicon film. a) top view, scale bar 300 nm and b) cross section, scale bar 2 mm.

FIG. 7 displays a schematic of the porous silicon biosensor concept (a, b) where antibody functionalised porous silicon captures luteinizing hormone. (c) Shows an anticipated shift of the photonic reflectance spectra from the porous silicon surface upon hormone binding and (d) photographs of the photonic porous silicon surfaces.

FIG. 8 shows the porous silicon modification method 1. In this case the porous silicon is first thermally hydrocarbonized (THC), with steps 1-8 showing thermal hydrosilylation with undecylenic acid, NHS ester activation, antibody attachment and polyethylene glycol (PEG) attachment respectively.

FIG. 9 shows the porous silicon modification method 2, where the surface is firstly thermally carbonized (TC), then functionalised with isocyanate silane (ICN) and finally antibody conjugation.

FIG. 10 shows the porous silicon modification method 3, where the surface is firstly thermally carbonized, then functionalised with mercaptopropyl silane (MPTMS), conjugated with PEG crosslinker and finally antibody conjugation.

FIG. 11 shows the LH detection and control scans for the porous silicon biosensor (functionalised by method 1), at an LH concentration of 100 μg/mL.

FIG. 12 shows the LH detection and control scans for the porous silicon biosensor (functionalised by method 1). A larger pore size has been used here and the LH concentration is 60 μg/mL.

FIG. 13 shows the LH detection and control scans for the porous silicon biosensor (functionalised by method 1). A larger pore size has been used here and the LH concentration is 1 μg/mL. In this case 8 mL of the LH solution was recirculated over the biosensor surface for a period of 15 hr.

FIG. 14 shows the maximum observed signals for LH detection at different concentrations.

FIG. 15 shows in the Left Panels: FDA stained cells showing cell morphology on two surfaces. Cells are rounded up on THC surface. Right Panels: Phase images showing cell morphologies, indicating conventional THC surfaces possesses certain degree of cytotoxicity.

FIG. 16 shows confocal fluorescence image of fibroblasts on tissue culture polystyrene (TCPS, flat silicon and pre-leached THC biosensor surface (PlTHC). The result indicates that fibroblast could adhere and spread normally on the PlTHC as if they are on TCP.

FIG. 17 shows confocal fluorescence images of fibroblasts on porous silicon with and without stimulation by TGFβ1. The result indicates that fibroblast could populate and form normal tissue on porous silicon biosensor.

FIG. 18 shows top view and cross section of control and TGFβ1 stimulated fibroblasts on porous silicon. The result indicates that stimulated cells form a thicker tissue, composed of more collagen 1. This more closely resembles an in vivo situation.

FIG. 19 shows the measured optical spectra from porous silicon rugate film covered with a fibrotic matrix, incubated in different concentrations of sucrose. The result indicates that the sucrose could diffuse through the induced fibrotic matrix, with subsequent detection successful even though the porous silicon film was covered with thick fibrotic tissue.

FIG. 20 shows a comparison of porous silicon rugate film response to different sucrose concentrations, with and without induced fibrotic matrix on the porous surface. The measured optical response indicates that the sensor functions normally, even with fibrotic tissue on the surface.

FIG. 21 shows two photographs of the biosensor implanted into a mouse (a) visible through the skin and (b) displaying reading the sensor with the fibre optic probe. (c) displays the measured spectra through the skin for weeks 1-4 post implant.

FIG. 22 shows the reflectance spectra from anti-mouse albumin functionalised pSi biosensor, pre-implant, implanted (through skin, weak signal), through removed skin section and post implant (biosensor surface removed from skin section). It can be seen that the signal of the post-implant surface has red shifted by approximately 30 nm, indicating protein binding within the pores.

FIG. 23 shows histological sections for sham, positive control and porous silicon biosensor implant sites.

FIG. 24 shows histological sections with sirius red staining of implant site for sham, PCL, THC and PlTHC. The results indicate that the amount of fibrotic tissue formation is very low for the PlTHC implant, which infers low inflammation in the tissue.

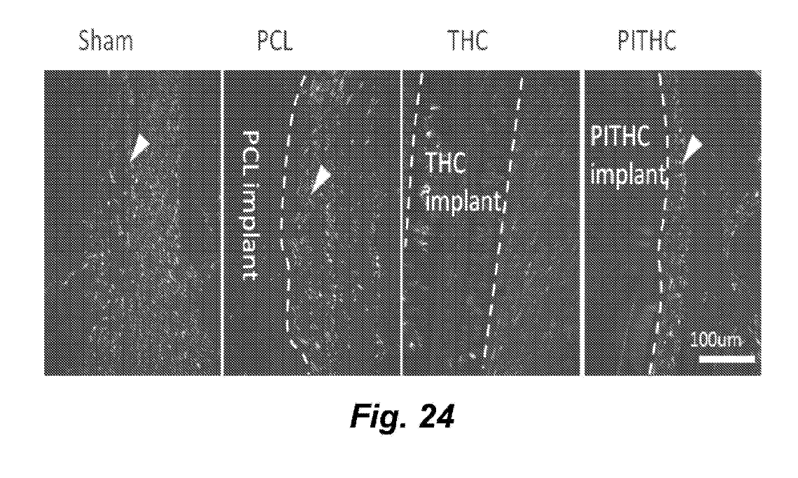

FIG. 25 shows histological sections for sham, positive control and porous silicon biosensor implant sites, stained against the inflammatory regulator IL-6.

FIG. 26 shows hematoxylin and eosin (H & E) staining of liver section from mice with PCL and porous silicon biosensor implant respectively. The results indicate that there is no toxicity induced by the biosensor.

FIG. 27 shows H & E staining of spleen section from mice with PCL and THC porous silicon biosensor and PlTHC biosensor implant respectively.

### DETAILED DESCRIPTION

The present disclosure relates to methods and sensors for detecting a biological parameter.

Certain disclosed embodiments have one or more combinations of advantages. For example, some of the advantages of the embodiments disclosed herein include one or more of the following: a method for improved detection of an implanted sensor; a method for improved detection of a sensor through the skin; a sensor with optical readout properties that allow signal detection through the skin; a sensor with improved biocompatability; a sensor composed of a biologically inert material; a sensor that breaks down in vivo to a product found in the body; a sensor with an optical output signal; a sensor whose readout characteristics alter on the binding of a molecule to the sensor; a sensor composed of a material with reduced inflammatory properties; a sensor that can be used to detect a biological parameter ex vivo, for example in milk and urine; to address one or more problems in the art; to provide one or more advantages in the art; and/or to provide a useful commercial choice. Other advantages of certain embodiments are disclosed herein.

The present disclosure is based upon the recognition that an optical signal between 400 and 1200 nm may used be for detecting a signal through the skin and that a sensor employing an interrogation signal and/or output signal between these wavelength ranges may be used to detect a biological parameter through the skin

Certain embodiments of the present disclosure provide a method of detecting a biological parameter.

Certain embodiments of the present disclosure provide a method of detecting a biological parameter using a sensor as described herein.

Certain embodiments of the present disclosure provide a method of detecting a biological parameter in a subject and/or in an organ, tissue or fluid derived from the subject.

Certain embodiments of the present disclosure provide a method of detecting a biological parameter in a subject and/or an organ, tissue or fluid derived from the subject, the method comprising using a sensor introduced into the subject or the organ, tissue or fluid derived from the subject to detect the biological parameter, wherein the sensor comprises an optical property between 400 and 1200 nm which is responsive to the biological parameter.

Certain embodiments of the present disclosure provide a method of detecting a biological parameter in a subject and/or an organ, tissue or fluid derived from the subject, the method comprising:
introducing a sensor into the subject or the organ, tissue or fluid derived from the subject;
detecting an optical property between 400 and 1200 nm from the introduced sensor, and
using the optical property to detect the biological parameter.

Certain embodiments of the present disclosure provide a method of detecting a biological parameter in a subject and/or an organ, tissue or fluid derived from the subject, the method comprising:
introducing a sensor into the subject or the organ, tissue or fluid derived from the subject, wherein the sensor comprises an optical property between 400 and 1200 nm which is responsive to the biological parameter;
detecting an optical property between 400 and 1200 nm from the introduced sensor, and
using the optical property to detect the biological parameter.

Examples of biological parameters include the presence, absence and/or concentration of an analyte, temperature, pH, oxygen level, carbon dioxide level, osmolarity and/or changes in any of the aforementioned parameters. Other types of biological parameters are contemplated.

In certain embodiments, the biological parameter is selected from the presence or absence of an analyte, the concentration of an analyte, a change in the concentration of an analyte, temperature, a change in temperature, pH, a change in pH, oxygen level, a change in oxygen level, carbon dioxide level, a change in carbon dioxide level, osmolarity and a change in osmolarity.

In certain embodiments, the biological parameter is the concentration of an analyte. In certain embodiments, the biological parameter is the change in concentration of an analyte.

Examples of analytes include one or more of a hormone, a growth factor, an antibody, an enzyme, a drug, a protein, a ligand, a nucleic acid, a non-nucleic acid molecule; a small molecule, a metabolite, a cofactor, an amino acid, a vitamin, a lipid, a carbohydrate, a sugar, a cell and/or a component thereof, an inflammatory marker, a toxin, a pesticide, a metal ion, a pathogen, a bacteria, a virus, an antigen, insulin, and an antibiotic. Other types of analytes are contemplated.

In certain embodiments, the analyte comprises a hormone. Examples of hormones include melatonin, serotonin, thyroxin, epinephrine, norepinephrine, popamine, antimullerian hormone, adiponectin, adrenocorticotropic hormone, angiotensinogen, antidiuretic hormone, atrial natriuretic peptide, calcitonin, cholecystokinin, corticotrophin-releasing hormone, erythropoietin, estrogen, follicle-stimulating hormone, gastrin, ghrelin, glucagon, growth hormone, human chorionic gonadotropin, growth hormone, insulin, insulin-like growth factor, leptin, luteinizing hormone, melanocyte stimulating hormone, orexin, oxytocin, parathyroid hormone, prolactin, secretin, aldosterone, testosterone, androstenedione, estradiol, progesterone, lipotropin, brain natriuretic peptide, histamine, endothelin, and enkephalin.

In certain embodiments, the hormone comprises a peptide-based hormone. In certain embodiments, the hormone comprises a steroid hormone.

In certain embodiments, the analyte comprises a hormone whose level is indicative of pregnancy. Examples of such hormones in humans include human chorionic gonadotropin (hCG), human chorionic somatolactotropin (hCS), steroid hormones such as oestrogen and progesterone, oxytocins, growth hormone, corticotropin-releasing hormone, proopiomelanocortin, prolactin and gonadotropin-releasing hormone. Examples of such hormones in cows include progesterone and estrone sulfate.

In certain embodiments, the analyte comprises a hormone whose level is indicative of ovulation status. Examples of such hormones in cows include luteinizing hormone, FSH and estradiol.

In certain embodiments, the hormone comprises luteinizing hormone.

In certain embodiments, the hormone comprises a size in the range from 25 to 40 kDa.

In certain embodiments, the method comprises detecting an analyte at a concentration of less than 1 mM, less than 1 μM, less than 1 nM, less than 1 μM, less than 1 fM or about less than one of the aforementioned values. In certain embodiments, the method comprises detecting an analyte at a concentration of 1 mM or less, 1 μM or less, 1 nM or less, 1 pM or less, 1 fM or less, or about one of the aforementioned values. In certain embodiments, the method comprises detecting an analyte at a concentration in a range selected from 1 mM to 1 μM, 1 mM to 1 nM, 1 mM to 1 pM, 1 mM to fM, 1 μM to 1 nM, 1 μM to 1 pM, 1 μM to fM, 1 nM to 1 pM, 1 nM to fM, 1 pM to 1 fM or about one of the aforementioned ranges.

In certain embodiments, the sensor comprises an interferometric sensor, an ellipsometric sensor, a field effect transistor based sensor, and/or a piezoresistive sensor. The use of such sensors is known in the art. Other types of sensors and detectors are contemplated.

In certain embodiments, the sensor comprises a porous silicon sensor. In certain embodiments, the sensor comprises one or more porous silicon layers.

In certain embodiments, the subject is human subject. In certain embodiments, the subject is an animal subject. In certain embodiments, the subject is a human subject or an animal subject.

In certain embodiments, the subject is a mammalian subject, a livestock animal (such as a horse, a cow, a sheep, a goat, a pig), a domestic animal (such as a dog or a cat) and other types of animals such as monkeys, rabbits, mice, birds and laboratory animals. Other types of animals are contemplated. Veterinary applications of the present disclosure are contemplated. Animal management applications of the present disclosure are contemplated. In certain embodiments, the animal is a bovine animal, an ovine animal, a porcine animal, an equine animal, and a caprine animal. Other types of animals and applications are contemplated.

In certain embodiments, the subject is a subject suitable for fertilisation, a subject suitable for insemination, or a subject that is pregnant or suitable for testing for pregnancy. For example, the methods of the present disclosure may be used to detect whether an animal subject, such as a livestock animal, is suitable for insemination or whether the animal is pregnant. Other uses are contemplated. Uses in human subjects are contemplated.

In certain embodiments, the subject is suffering from, or susceptible to, a disease, condition or state. Diagnostic and prognostic applications are contemplated.

In certain embodiments, the detecting of the biological parameter comprises detection of the parameter in vivo.

In certain embodiments, the detecting of the biological parameter comprises detection of the parameter ex vivo. In certain embodiments, the detecting of the biological parameter includes detection of the parameter in an organ, tissue or fluid derived from the subject.

In this regard, the term “derived” refers to detecting of the biological parameter in an organ, tissue or fluid removed from the subject, detecting the biological parameter in a sample obtained from an organ, tissue or fluid and/or in a processed and/or treated form thereof. For example, the sample may be a derivative, an extract, a treated form, pre-cleared, filtered, desalted, concentrated, diluted, buffered, centrifuged, induced, pre-treated, processed to remove one or more components or impurities from the sample, or suitable combinations thereof. Other forms of processing and/or treatment are contemplated.

In certain embodiments, the detecting of the biological parameter comprises detection of the parameter in vitro.

In certain embodiments, the method comprises detecting the biological parameter in vivo.

Certain embodiments of the present disclosure provide a method of detecting a biological parameter in a subject in vivo, the method comprising:
introducing a sensor into the subject;
detecting an optical property between 400 and 1200 nm from the introduced sensor, and
using the optical property to detect the biological parameter in vivo.

In certain embodiments, the method comprises detecting the biological parameter in a body fluid ex vivo.

In certain embodiments, the method comprises detecting the biological parameter in a fluid from the subject, such as milk, colostrum, blood or urine. In certain embodiments, the method comprises detecting the biological parameter in a biological fluid and/or a processed form thereof. For example, the biological fluid may be treated, pre-cleared, filtered, desalted, concentrated, diluted, buffered, centrifuged, induced, pre-treated, or otherwise processed to remove one or more components or impurities, or suitable combinations of any of the aforementioned. Other forms of processing or treatment are contemplated.

In certain embodiments, the detecting of the biological parameter comprises unassisted detection. In certain embodiments, the detecting of the biological parameter comprises assisted detection, such as using a reflectance probe and/or other components, systems or devices.

In certain embodiments, the detecting of the biological parameter comprises detection using the unaided eye. For example, a change in colour may be used to detect the biological parameter.

In certain embodiments, the detecting of the biological parameter comprises a visual detection. In certain embodiments, the detecting of the biological parameter comprises a read-out of information, such as an optical signature.

In certain embodiments, the detecting of the biological parameter comprises a qualitative detection. For example, a change in colour using the unaided eye or other type of detector may provide qualitative detection.

In certain embodiments, the sensor provides quantitative detection. In certain embodiments, the sensor provides qualitative and/or quantitative detection.

In certain embodiments, the detecting of the biological parameter comprises detecting the presence or absence of an analyte. In certain embodiments, the detecting of the biological parameter comprises detecting the location of an analyte. In certain embodiments, the detecting of the biological parameter comprises determining the concentration or level of an analyte. In certain embodiments, the detecting of the biological parameter comprises determining the change in concentration or level of an analyte.

In certain embodiments, the optical property comprises an optical output signal. In certain embodiments, the optical property comprises an optical output signal which is responsive to the binding of an analyte.

In certain embodiments, the optical property comprises an optical reflectance property. In certain embodiments, the optical property comprises an optical reflectance property which is responsive to the biological parameter. In certain embodiments, the optical property comprises an optical reflectance property which is responsive to the binding of an analyte.

In certain embodiments, the method comprises detecting an optical property between 400 and 1200 nm, or about the aforementioned range. In certain embodiments, the method comprises detecting an optical property between 600 nm and 800 nm, or about the aforementioned range.

In certain embodiments, the method comprises detecting an optical property in a range selected from 500 to 600 nm, 550 to 750 nm, 600 to 800 nm, 650 to 800 nm, or 800 to 1200 nm, or one of the aforementioned ranges.

In certain embodiments, the method comprises detecting an optical property in one of the selected ranges: 400 to 1200 nm, 400 to 1100 nm, 400 to 1000 nm, 400 to 900 nm, 400 to 800 nm, 400 to 700 nm, 400 to 600 nm, 400 to 500 nm, 500 to 1200 nm, 500 to 1100 nm, 500 to 1000 nm, 500 to 900 nm, 500 to 800 nm, 500 to 700 nm, 500 to 600 nm, 600 to 1200 nm, 600 to 1100 nm, 600 to 1000 nm, 600 to 900 nm, 600 to 800 nm, 600 to 700 nm, 700 to 1200 nm, 700 to 1100 nm, 700 to 1000 nm, 700 to 900 nm, 700 to 800 nm, 800 to 1200 nm, 800 to 1100 nm, 800 to 1000 nm, 800 to 900 nm, 900 to 1200 nm, 900 to 1100 nm, 900 to 1000 nm, 1000 to 1200 nm, 1000 to 1100 nm, to 1100 to 1200 nm, or about one of the aforementioned ranges. Other ranges are contemplated.

In certain embodiments, the optical property comprises a wavelength spectrum. In certain embodiments, the optical property comprises one or more wavelength ranges.

In certain embodiments, the optical property comprises one or more discrete wavelengths.

In certain embodiments, the optical property comprises one or more reflectance wavelength ranges and/or one or more discrete reflectance wavelengths.

In certain embodiments, the optical property comprises an optical reflectance property.

In certain embodiments, the optical reflectance property comprises an optical reflectance property between 600 nm and 800 nm, or about the aforementioned. In certain embodiments, the method comprises detecting an optical reflectance property between 600 nm and 800 nm, or about the aforementioned range.

In certain embodiments, the optical reflectance property comprises an optical reflectance property in a range selected from 500 to 600 nm, 550 to 750 nm, 600 to 800 nm, 650 to 800 nm or 800 to 1200 nm, or about one of the aforementioned ranges.

In certain embodiments, the optical reflectance property comprises an optical reflectance property in one of the selected ranges: 400 to 1200 nm, 400 to 1100 nm, 400 to 1000 nm, 400 to 900 nm, 400 to 800 nm, 400 to 700 nm, 400 to 600 nm, 400 to 500 nm, 500 to 1200 nm, 500 to 1100 nm, 500 to 1000 nm, 500 to 900 nm, 500 to 800 nm, 500 to 700 nm, 500 to 600 nm, 600 to 1200 nm, 600 to 1100 nm, 600 to 1000 nm, 600 to 900 nm, 600 to 800 nm, 600 to 700 nm, 700 to 1200 nm, 700 to 1100 nm, 700 to 1000 nm, 700 to 900 nm, 700 to 800 nm, 800 to 1200 nm, 800 to 1100 nm, 800 to 1000 nm, 800 to 900 nm, 900 to 1200 nm, 900 to 1100 nm, 900 to 1000 nm, 1000 to 1200 nm, 1000 to 1100 nm, to 1100 to 1200 nm, or about one of the aforementioned ranges. Other ranges are contemplated.

In certain embodiments, the optical reflectance property comprises a reflectance wavelength spectrum. In certain embodiments, the optical reflectance property comprises one or more reflectance wavelength ranges. In certain embodiments, the optical reflectance property comprises one or more discrete reflectance wavelengths.

In certain embodiments, the optical reflectance property comprises one or more reflectance wavelength ranges and/ or one or more discrete reflectance wavelengths.

In certain embodiments, the optical reflectance property comprises a photonic peak of the optical reflectance property. In certain embodiments, the optical reflectance property comprises a change of the photonic peak of the optical reflectance property.

In certain embodiments, the change of the photonic peak comprises an increase in the wavelength of the photonic peak.

In certain embodiments, the change of the photonic peak is indicative of the value of the biological parameter. In certain embodiments, the change of the photonic peak is indicative of the value of the biological parameter in the subject.

In certain embodiments, the detecting of the optical property comprises visual detection of the optical property. In certain embodiments, the detecting of the optical property comprises visual detection to the unaided eye of the optical property. In certain embodiments, the detecting of the optical property comprises spectrographic detection of the optical property. Other methods of detection are contemplated.

In certain embodiments, the detecting of the optical reflectance property comprises visual detection of the optical reflectance property. In certain embodiments, the detecting of the optical reflectance property comprises spectrographic detection of the optical reflectance property.

In certain embodiments, the method comprises introducing the sensor into the subject or an organ, tissue or fluid derived from the subject, as described herein.

In certain embodiments, the method comprising implanting the sensor into the subject. In certain embodiments, the method comprises introducing the sensor under/below the skin of the subject. In certain embodiments, the method comprises implanting a sensor under/below the skin of the subject. In certain embodiments, the method comprises introducing the sensor under/below the dermis of the subject. In certain embodiments, the method comprises implanting the sensor under/below the dermis of the subject. In certain embodiments, the method comprises implanting the sensor under/below the epidermis of the subject.

Certain embodiments of the present disclosure provide a method of detecting a biological parameter in a subject, the method comprising:
implanting a sensor into the subject, wherein the sensor comprises an optical property between 400 and 1200 nm which is responsive to the biological parameter;
detecting an optical property between 400 and 1200 nm through the skin of the subject from the implanted sensor, and
using the optical property to detect the biological parameter.

Certain embodiments of the present disclosure provide a method of detecting a biological parameter in a subject, the method comprising:
implanting a sensor under the skin of the subject, wherein the sensor comprises an optical property between 400 and 1200 nm which is responsive to the biological parameter;
detecting an optical property between 400 and 1200 nm through the skin of the subject from the implanted sensor, and
using the optical property to detect the biological parameter.

Certain embodiments of the present disclosure provide a method of detecting a biological parameter in a subject, the method comprising:
implanting a sensor into the subject, wherein the sensor comprises an optical reflectance property between 400 and 1200 nm which is responsive to the biological parameter;
detecting an optical reflectance property between 400 and 1200 nm through the skin of the subject from the implanted sensor, and
using the optical reflectance property to detect the biological parameter

Certain embodiments of the present disclosure provide a method of detecting a biological parameter in a subject, the method comprising:
implanting a sensor under the skin of the subject, wherein the sensor comprises an optical reflectance property between 400 and 1200 nm which is responsive to the biological parameter;
detecting an optical reflectance property between 400 and 1200 nm through the skin of the subject from the implanted sensor, and
using the optical reflectance property to detect the biological parameter

In certain embodiments, the sensor is implanted into an organ or tissue in the subject.

In certain embodiments, the sensor is introduced less than 1 cm below the skin, or about less than 1 cm less below the skin. In certain embodiments, the sensor is introduced greater than 1 cm below the skin. Typically, the sensor is introduced 1 to 3 mm below the skin, or about 1 to 3 cm below the skin. In certain embodiments, the sensor is introduced 1 cm or less below the skin, or about 1 cm or less below the skin. In certain embodiments, the sensor is introduced 2 cm or less below the skin, or about 2 cm or less below the skin. In certain embodiments, the sensor is introduced 3 cm or less below the skin, or about 3 cm or less below the skin. In certain embodiments, the sensor is introduced 1 cm or greater below the skin, or about 1 cm or greater below the skin. In certain embodiments, the sensor is introduced 2 cm or greater below the skin, or about 2 cm or greater below the skin. In certain embodiments, the sensor is introduced 3 cm or greater below the skin, or about 3 cm or greater below the skin.

In certain embodiments, the method comprises detecting the optical property through the skin. In certain embodiments, the method comprises detecting the optical reflectance property through the skin.

For example, in dairy cows the sensor may be introduced into the udder, which allows detection of the biological parameter during milking, if so desired. Examples of other sites of introduction include the ear, the nose, the neck, the rump, and the tail.

In certain embodiments, the sensor comprises an analyte binding molecule.

In certain embodiments, the sensor comprises an analyte binding molecule and and the optical property changes upon binding of the analyte to the analyte binding molecule. In certain embodiments, the sensor comprises an analyte binding molecule and the optical property is responsive to binding of the analyte to the analyte binding molecule.

In certain embodiments, the sensor comprises an analyte binding molecule and the optical reflectance property changes upon binding of the analyte to the analyte binding molecule. In certain embodiments, the sensor comprises an analyte binding molecule and the optical reflectance property is responsive to binding of the analyte to the analyte binding molecule.

In certain embodiments, the analyte binding molecule comprises one or more of an antibody and/or an antigen binding fragment thereof, a receptor, a ligand, an aptamer, a nucleic acid, a protein, a small molecule, a drug, a co-factor, a virus and/or a part thereof, a carbohydrate, and a lipid. In this regard, the analyte binding molecule is typically a molecule that may bind the analyte with high specificity and/or affinity and thereby produces a change in an optical property from the sensor upon binding.

In certain embodiments, the sensor comprises an analyte binding molecule and the optical property of the sensor changes upon binding of the analyte to the analyte binding molecule.

In certain embodiments, the analyte binding molecule is attached to the sensor. In certain embodiments, the analyte binding molecule is covalently linked to the sensor. In certain embodiments, the analyte binding molecule is non-covalently linked to the sensor. Methods for linking of an analyte binding molecule to a sensor are described herein.

In certain embodiments, the analyte binding molecules comprises an antibody and/or an antigen binding fragment thereof. Other types of binding pairs are contemplated.

The term “antibody” refers to an immunoglobulin molecule with the ability to bind an antigenic region of another molecule, and includes monoclonal antibodies, polyclonal antibodies, multivalent antibodies, chimeric antibodies, multispecific antibodies, diabodies and fragments of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

In certain embodiments, the antibody and/or an antigen binding fragment thereof is attached to the sensor through a carbohydrate group on the antibody and/or antigen binding fragment thereof. In certain embodiments, the antibody and/or the antigen binding fragment thereof is attached to the sensor via the Fc fragment (if present). In certain embodiments, antibody and/or the antigen binding fragment thereof comprises an oxidized carbohydrate group for attachment. In certain embodiments, the antibody and/or the antigen binding fragment thereof is attached via an oxidized carbohydrate group.

In certain embodiments, the analyte comprises a hormone and the analyte binding molecule comprises an antibody to the hormone and/or an antigen binding fragment thereof.

In certain embodiments, the analyte comprises luteinizing hormone and the analyte binding molecule comprises an antibody to luteinizing hormone and/or an antigen binding fragment thereof. Anti-luteinizing hormone antibodies, including monoclonal antibodies, are known in the art and/or commercially available.

In certain embodiments, the antibody and/or antigen binding fragment thereof has a thermodynamic dissociation constant Kd for dissociation from its target of equal to, or greater than, 10⁻⁷M, 10⁻⁸M, 10⁻⁹M, 10⁻¹⁰M or 10⁻¹¹M. In certain embodiments, the antibody and/or antigen binding fragment thereof has a thermodynamic dissociation Kd of 10⁻⁷M or greater, 10⁻⁸M or greater, 10⁻⁹M or greater, 10⁻¹⁰or greater, or 10⁻¹¹M or greater. In certain embodiments, the Kd is in the range from 10⁻⁸M to 10⁻¹²M.

In certain embodiments, the sensor comprises one or more porous silicon layers, as described herein. In certain embodiments, the sensor comprises multiple porous silicon layers.

In certain embodiments, the one or more of the porous silicon layers comprise an analyte binding molecule and the optical property changes upon binding of the analyte to the analyte binding molecule. In certain embodiments, the one or more of the porous silicon layers comprise an analyte binding molecule and the optical property is responsive to binding of the analyte to the analyte binding molecule.

In certain embodiments, the one or more of the porous silicon layers comprise an analyte binding molecule and the optical reflectance property changes upon binding of the analyte to the analyte binding molecule. In certain embodiments, the one or more of the porous silicon layers comprise an analyte binding molecule and the optical reflectance property is responsive to binding of the analyte to the analyte binding molecule.

In certain embodiments, the analyte binding molecule is attached to one or more of the porous silicon layers. In certain embodiments, the analyte binding molecule is covalently linked to one or more of the porous silicon layers. In certain embodiments, the analyte binding molecule is non-covalently linked to one or more of the porous silicon layers.

In certain embodiments, the sensor comprises a plurality of porous silicon layers. Methods for producing a sensor comprising a plurality of porous silicon layers are as described herein.

In certain embodiments, the sensor comprises a single layer porous silicon layer. Methods for producing a sensor comprising a single porous silicon layer are as described in N. H. Voelcker, I. Alfonso, M. R. Ghadiri. Catalyzed Oxidative Corrosion of Porous Silicon Used as an Optical Transducer for Ligand-Receptor Interaction. Chem Bio Chem 9 (2008), 1176-1786.

In certain embodiments, the plurality of porous silicon layers comprise a Bragg reflector. In certain embodiments, the plurality of porous silicon layers comprise a rugate filter.

In certain embodiments, the sensor comprises microcavities between porous silicon layers.

In certain embodiments, the sensor comprises a film or membrane comprising the one or more silicon layers. Methods for producing a sensor comprising a film or membrane comprising one or more silicon layers are known in the art. In certain embodiments, the sensor comprises particles comprising the one or more silicon layers. Methods for producing a sensor comprising particles comprising one or more silicon layers are known in the art.

In certain embodiments, the sensor comprises an optical fibre. Methods for using optical fibres to transmit optical signals are known in the art.

In certain embodiments, the sensor comprises an optical property that is indicative of the identity of the subject. In this regard, the optical properties of the sensor can, if so desired, be tailored to provide a signature that is indicative of the identity of one or more subjects. For example, animals can have a sensor as described herein implanted and the sensor can be used to tag the subject. For example, a sensor may have a unique optical signature and as such can be used to match the optical signature with an individual animal.

In certain embodiments, the sensor comprises an optical reflectance property that is indicative of the identity of the subject.

In certain embodiments, the sensor is substantially biocompatible. In certain embodiments, the sensor is pre-treated to improve biocompatibility. In certain embodiments, the sensor is exposed to one or more biological molecules to improve biocompatibility, such as pre-treatment with a tissue culture medium.

In certain embodiments, the one or more porous silicon layers are pre-treated to improve biocompatibility. In certain embodiments, the one or more silicon layers are exposed to one or more biological molecules to improve biocompatibility, such as pre-treatment with a tissue culture medium.

In certain embodiments, the sensor comprises an in vivo half life of less than 2 weeks. In certain embodiments, the sensor comprises an in vivo half life of greater than 2 weeks. In certain embodiments, the sensor comprises an in vivo half life of less than 4 months. In certain embodiments, the sensor comprises an in vivo half life of greater than 4 months. In certain embodiments, the sensor comprises an in vivo half life of 2 weeks to 4 months. In certain embodiments, the sensor comprises a half of about one of the aforementioned half-lives. Other half lives are contemplated.

In certain embodiments the one or more porous silicon layers comprise pores of a size of 5 to 500 nm. In certain embodiments the one or more porous silicon layers comprise pores of a size of 10 to 500 nm.

In certain embodiments, the one or more porous silicon layers comprise a pore size of 5 to 500 nm, 5 to 400 nm, 5 to 300 nm, 5 to 200 nm, 5 to 100 nm, 10 to 500 nm, 10 to 400 nm, 10 to 300 nm, 10 to 200 nm or 10 to 100 nm. In certain embodiments, the porous silicon particles comprise a pore size of 500 nm or less, 400 nm or less, 300 nm or less, 200 nm or less, 100 nm or less, or 50 nm or less. In certain embodiments, the porous silicon particles comprise a pore size of at least 5 nm, at least 10 nm, at least 20 nm, at least 50 nm, or at least 100 nm. Methods for determining the pore size are known in the art.

In certain embodiments, the method as described herein may be used to determine whether a subject is ovulating, to determine whether a subject is pregnant, to determine the health of a subject, to determine whether a subject is suffering from or susceptible to a disease, condition or state, to determine whether a subject is in need of treatment, and to determine the health and/or fitness of a subject. Other uses are contemplated.

Certain embodiments of the present disclosure provide a method of determining the ovulation status of a subject, the method comprising:
implanting into the subject a sensor comprising one or more porous silicon layers and a luteinizing hormone binding molecule, wherein the sensor comprises an optical reflectance property between 400 and 1200 nm which is responsive to binding of leutinizing hormone to the sensor;
detecting an optical reflectance property between 400 and 1200 nm through the skin of the subject from the implanted sensor; and
determining the ovulation status of the subject on the basis of the optical reflectance property detected.

Certain embodiments of the present disclosure provide a method of determining the pregnancy status of a subject, the method comprising:
implanting into the subject a sensor comprising one or more porous silicon layers and a molecule that binds to an analyte indicative of pregnancy status, wherein the sensor comprises an optical reflectance property between 400 and 1200 nm which is responsive to binding of the analyte to the sensor;
detecting an optical reflectance property between 400 and 1200 nm through the skin of the subject from the implanted sensor; and
determining the pregnancy status of the subject on the basis of the optical reflectance property detected.

Certain embodiments of the present disclosure provide a method of determining the ovulation status of a subject, the method comprising:
implanting into the subject a sensor comprising one or more porous silicon layers and an anti-luteinizing hormone antibody, wherein the sensor comprises an optical reflectance property between 400 and 1200 nm which is responsive to binding of leutinizing hormone to the sensor;
detecting an optical reflectance property between 400 and 1200 nm through the skin of the subject from the implanted sensor; and
determining the ovulation status of the subject on the basis of the optical reflectance property detected.

Certain embodiments of the present disclosure provide a method of determining the pregnancy status of a subject, the method comprising:
implanting into the subject a sensor comprising one or more porous silicon layers and an antibody to an analyte indicative of pregnancy status, wherein the sensor comprises an optical reflectance property between 400 and 1200 nm which is responsive to binding of the analyte to the sensor;
detecting an optical reflectance property between 400 and 1200 nm through the skin of the subject from the implanted sensor; and
determining the pregnancy status of the subject on the basis of the optical reflectance property detected.

Certain embodiments of the present disclosure provide a sensor for detecting a biological parameter.

Sensors are as described herein. Certain embodiments of the present disclosure provide use of a sensor as describe herein for detecting a biological parameter.

Certain embodiments of the present disclosure provide a sensor for detecting a biological parameter, the sensor comprising an optical property between 400 and 1200 nm (or about this range) that is responsive to the biological parameter. Sensors are as described herein. Optical properties are as described herein.

Certain embodiments of the present disclosure provide a sensor for detecting a biological parameter, the sensor comprising an optical reflectance property between 400 and 1200 nm (or about this range) that is responsive to the biological parameter. Optical reflectance properties are as described herein.

In certain embodiments, the sensor provides qualitative sensing. In certain embodiments, the sensor provides quantitative sensing. In certain embodiments, the sensor provides qualitative and/or quantitative sensing.

In certain embodiments, the sensor comprises a silicon substrate. Methods for producing silicon substrates are known in the art.

In certain embodiments, the sensor comprises one or more of a flat substrate, a film, a membrane and particles.

In certain embodiments, the sensor comprises a flat silicon substrate. Methods for producing flat silicon are known in the art. In certain embodiments, the sensor comprises a silicon film or membrane. Methods for producing silicon films or membranes are known in the art.

In certain embodiments, the sensor comprises silicon particles. Methods for producing silicon particles are known in the art. For example, porous silicon particles of the desired size may be produced from porous silicon wafers or from free-standing porous silicon membranes using a controlled ultrasonication process. In certain embodiments, the particles comprise a size in the range of 10 to 1000 μm, or a size about this range. Other sizes are contemplated. Methods for determining the size of silicon particles are known in the art.

In certain embodiments, the sensor comprises a porous substrate. In certain embodiments, the sensor comprises a porous silicon substrate. Methods for producing porous silicon are known in the art and are as described herein.

In certain embodiments, the sensor comprises a porous aluminium substrate. Porous aluminium is known in the art. In certain embodiments, the substrate comprises a flat gold substrate. Flat gold is known in the art. In certain embodiments, the substrate comprises a porous silver substrate. Methods for producing porous substrates as described herein are known in the art.

In certain embodiments, the sensor comprises a porosity of at least 50%. Methods for determining porosity are known in the art.

In certain embodiments, the sensor comprises a porosity of at least 50%, at least 60%, at least 70%, or at least 90%. In certain embodiments, the sensor comprises a porosity of 90% or less, 80% or less, 70% or less, or 60% or less. In certain embodiments, the sensor comprises a porosity of 50 to 90%, 60 to 90%, 70 to 90%, 80 to 90%, 50 to 80%, 60 to 80%, 70 to 80%, 50 to 70%, 60 to 70%, or 50 to 60%. Methods for determining porosity are known in the art.

In certain embodiments, the sensor comprises a pore size of 5 to 500 nm. In certain embodiments, the sensor comprises a pore size of 10 to 500 nm. Pore sizes are as described herein. Methods for determining the pore size are known in the art.

In certain embodiments, the sensor comprises a functionalised substrate. In certain embodiments, the sensor comprises a functionalised sensor. The term “functionalising” and related terms refers to the addition of one or more chemical groups directly and/or indirectly to the surface of a substrate. Methods for functionalising substrates are known in the art. In certain embodiments, the sensor comprises a functionalised silicon substrate.

In certain embodiments the sensor comprises a silicon substrate. In certain embodiments, the functionalising comprises hydrosilylation of the silicon substrate. In certain embodiments, the functionalising comprises electrografting of the silicon substrate. In certain embodiments, the functionalising comprises oxidation of the silicon substrate. In certain embodiments, the functionalising comprises silanisation of the silicon substrate. In certain embodiments, the functionalising comprises hydrosilylation and/or silanisation of the silicon substrate.

In certain embodiments, the functionalising comprises dual hydrosilyation.

In certain embodiments, the functionalising comprises addition of a reactive linker to the sensor.

Certain embodiments of the present disclosure provide a sensor for detecting a biological parameter, the sensor comprising one or more porous silicon layers, wherein the sensor comprises an optical property between 400 and 1200 nm that is responsive to the biological parameter.

Certain embodiments of the present disclosure provide a sensor for detecting a biological parameter, the sensor comprising one or more porous silicon layers, wherein the sensor comprises an optical reflectance property between 400 and 1200 nm that is responsive to the biological parameter.

Optical properties, including optical reflectance properties, are as described herein.

In certain embodiments, the sensor comprises an optical reflectance property between 650 nm and 800 nm. In certain embodiments, the sensor comprises an optical reflectance property between 550 nm and 750 nm.

In certain embodiments, the optical reflectance property comprises a photonic peak of the optical reflectance property. In certain embodiments, the optical reflectance property comprises a change in photonic peak of the optical reflectance property.

Biological parameters are as described herein.

In certain embodiments, the biological parameter is selected from the presence or absence of an analyte, the concentration of an analyte, a change in the concentration of an analyte, temperature, change in temperature, pH, a change in pH, oxygen level, a change in oxygen level, carbon dioxide level, a change in carbon dioxide level, osmolarity and a change in osmolarity.

Analytes are as described herein.

In certain embodiments, the analyte comprises one or more of a hormone, a growth factor, an antibody, an enzyme, a drug, a protein, a ligand, a nucleic acid, a small molecule, a metabolite, a cofactor, an amino acid, a vitamin, a lipid, a carbohydrate, a sugar, a cell and/or a component thereof, an inflammatory marker, a toxin, a pesticide, a metal ion, a pathogen, a bacteria, a virus, an antigen, insulin, and an antibiotic. Other analytes are contemplated.

In certain embodiments, the sensor comprises an analyte binding molecule as described herein. In certain embodiments, the sensor comprises an analyte binding molecule and the optical property of the sensor changes upon binding of the analyte to the analyte binding molecule. In certain embodiments, the sensor comprises an analyte binding molecule and the optical reflectance property of the sensor changes upon binding of the analyte to the analyte binding molecule.

In certain embodiments, the analyte binding molecule is covalently linked to the sensor, as described herein.

Analyte binding molecules are as described herein. In certain embodiments, the analyte binding molecule comprises one or more of an antibody and/or an antigen binding fragment thereof, a receptor, a ligand, an aptamer, a nucleic acid, a protein, a small molecule, a drug, a co-factor, a virus and/or a part thereof, a carbohydrate, and a lipid. Other analyte binding molecules are contemplated.

In certain embodiments, the analyte binding molecule comprises an antibody and/or an antigen binding fragment thereof. Antibodies, and antigen binding fragments thereof, are as describe herein.

In certain embodiments, the analyte comprises a hormone whose level is indicative of pregnancy. In certain embodiments, the analyte comprises a hormone whose level is indicative of ovulation status. In certain embodiments, the hormone comprises luteinizing hormone. Examples of hormones are as described herein.

In certain embodiments, the analyte comprises a hormone and the analyte binding molecule comprises an antibody to the hormone and/or an antigen binding fragment thereof. Antibodies, and antigen binding fragments thereof, to hormones are as describe herein.

In certain embodiments, the analyte comprises luteinizing hormone and the analyte binding molecule comprises an antibody to luteinizing hormone and/or an antigen binding fragment thereof.

In certain embodiments, the sensor comprises one or more porous silicon layers, as described herein.

In certain embodiments, the one or more of the porous silicon layers comprise an analyte binding molecule and the optical reflectance property changes upon binding of the analyte to the analyte binding molecule, as described herein.

In certain embodiments, the analyte binding molecule is adsorbed to the sensor. Methods for adsorbing agents to substrates are known. In certain embodiments, the analyte binding molecule is physically adsorbed. In certain embodiments, the analyte binding agent is passively adsorbed. In certain embodiments, the sensor comprises passively adsorbed analyte binding molecule. In certain embodiments, the analyte binding molecule is actively adsorbed to the sensor.

In certain embodiments, the analyte binding molecule is attached to the sensor. For example, an analyte binding molecule may be attached to one or more porous silicon layers. In certain embodiments, the one or more porous silicon layers comprise an attached analyte binding molecule. The analyte binding molecule may be directly or indirectly attached.

In certain embodiments, the analyte binding molecule is non-covalently attached to the sensor. For example, the one or more porous silicon layers may comprise a non-covalently attached analyte binding molecule. Methods for non-covalent attachment are known in the art. In certain embodiments, the analyte binding molecule is non-covalently linked to the one or more porous silicon layers. Methods for non-covalent linking are known in the art.

In certain embodiments, the analyte binding molecule is covalently linked to the sensor. For example, the analyte binding molecule may be linked to one or more porous silicon layers by a cleavable chemical linker. In certain embodiments, the one or more porous silicon layer comprise an analyte binding covalently linked via a cleavable chemical linker. Cleavable chemical linkers are known in the art. Examples of cleavable linkers include disulfides, o-nitrobenzyls, esters, carbamates, acetals, orthoesters, trityls, ketals, imines, vinyl ethers and hydrazones.

Analyte binding molecules are as described herein. In certain embodiments, the analyte binding molecule comprises an antibody and/or a binding fragment thereof.

In certain embodiments, the analyte binding molecule comprises an antibody and/or an antigen binding fragment thereof. In certain embodiments, the antibody and/or an antigen binding fragment thereof is attached to the sensor through a carbohydrate group on the antibody and/or antigen binding fragment thereof. In certain embodiments, the antibody and/or the antigen binding fragment thereof is attached to the sensor via the Fc fragment. In certain embodiments, antibody and/or the antigen binding fragment thereof comprises an oxidized carbohydrate group. In certain embodiments, the antibody and/or the antigen binding fragment thereof is attached via an oxidized carbohydrate group.

The term “antibody” refers to an immunoglobulin molecule with the ability to bind an antigenic region of another molecule, and includes monoclonal antibodies, polyclonal antibodies, multivalent antibodies, chimeric antibodies, multispecific antibodies, diabodies and fragments of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

In certain embodiments, the antibody is a monoclonal antibody and/or an antigen binding fragment thereof, as described herein. In certain embodiments, the antibody is a human antibody or a humanized antibody. In certain embodiments, the antibody is a bovine antibody.

In certain embodiments, the analyte binding molecule is loaded onto the sensor. For example, the analyte binding molecule may be passively loaded by absorption of the analyte binding molecule into one or more porous silicon layers. The analyte binding molecule may also, for example, be actively loaded by chemical coupling to the sensor directly or indirectly, as described herein.

In certain embodiments, the analyte binding molecule is attached to the sensor. In certain embodiments, the analyte binding molecule is directly attached to the sensor. In certain embodiments, the analyte binding molecule is indirectly attached.

In certain embodiments, the analyte binding molecule is covalently attached to the sensor. In certain embodiments, the sensor comprises a covalently attached analyte binding molecule.

In certain embodiments, the analyte binding molecule is non-covalently attached to the sensor. In certain embodiments, the sensor comprises a non-covalently attached analyte binding molecule.

In certain embodiments, the analyte binding molecule is attached to the sensor via a linker. In certain embodiments, the sensor comprises an analyte binding molecule attached via a linker. Examples of linkers include carboxyl-to-amine linkers, such as carbodiimides, amine-reactive linkers such as NHS esters and imidoesters, sulfhydryl-reactive linkers such as maleimides, haloacetyls and pyridyldisulfides, carbonyl-reactive linkers such as hydrazides and alkoxyamines, photoreactive linkers such as aryl azides and diazirines, chemoselective ligation, such as Staudinger reagent pairs. In certain embodiments, the linker comprises a semicarbazide linker. Other linkers are contemplated.

In certain embodiments, the analyte binding molecule is an antibody. In certain embodiments, the antibody is attached (directly or indirectly) to the sensor via at least the Fc chain of the antibody.

In certain embodiments, the sensor comprises a single layer porous silicon layer. Methods for producing a sensor with a single layer of porous silicon are known in the art.

In certain embodiments, the sensor comprises a plurality of porous silicon layers. In certain embodiments, the plurality of porous silicon layers comprise a Bragg reflector. In certain embodiments, the plurality of porous silicon layers comprise a rugate filter.

In certain embodiments, the sensor comprises microcavities between porous silicon layers.

In certain embodiments, the porous silicon layers are electro-chemically etched into the silicon layer by a process utilising a composite-current time waveform.

In certain embodiments, the sensor comprises a distinguishable optical property. In this regard, such a sensor can be used to tag one or more subjects so that the optical property is indicative of the identity of a subject. In certain embodiments, the sensor comprises a distinguishable optical reflectance property.

In certain embodiments, the sensor comprises a membrane comprising one or more silicon layers.

In certain embodiments, the sensor comprises particles comprising one or more silicon layers.

In certain embodiments, the sensor comprises an optical fibre. Certain embodiments of the present disclosure provide a sensor as described herein comprising an optical fibre.

Methods for incorporating sensors into, or in communication with, optical fibres are known in the art. For example, an optically reflective sensor or substrate as described herein may be used in conjunction with an optical fibre, thereby allowing changes in the reflective properties of the substrate to be measured at a site using the optical fibre. In this way, a parameter can be assessed or measured.

In certain embodiments, the sensor is substantially biocompatible. In certain embodiments, the sensor is pre-treated to improve biocompatibility. In certain embodiments, the sensor is exposed to one or more biological molecules to improve biocompatibility, such as pre-treatment with a tissue culture medium, as described herein.

In certain embodiments, the one or more porous silicon layers are pre-treated to improve biocompatibility. In certain embodiments, the one or more silicon layers are exposed to one or more biological molecules to improve biocompatibility, such as pre-treatment with a tissue culture medium.

Examples of the in vivo half life of the sensor are as described herein. In certain embodiments, the sensor comprises an in vivo half life of 2 weeks to 4 months, or an in vivo half life of about this range.

Certain embodiments of the present disclosure provide a method of detecting a biological parameter, the method comprising using a sensor as described herein to detect the biological parameter.

In certain embodiments, the biological parameter is a biological parameter in vivo. In certain embodiments, the biological parameter is a biological parameter ex vivo. In certain embodiments, the biological parameter is a biological parameter in vitro.

In certain embodiments, the sensor is implanted into a subject and the optical reflectance property of the implanted sensor is detected through the skin of the subject. In certain embodiments, the sensor is implanted under the skin of a subject and the optical reflectance property of the implanted sensor is detected through the skin of the subject.

Certain embodiments of the present disclosure provide a method of detecting a biological parameter, the method comprising using a sensor to detect the biological parameter, wherein the sensor comprises an optical property between 400 and 1200 nm (or about this range) that is responsive to the biological parameter.

Certain embodiments of the present disclosure provide a method of detecting a biological parameter, the method comprising using a sensor comprising one or more porous silicon layers to detect the biological parameter, wherein the sensor comprises an optical reflectance property between 400 and 1200 nm (or about this range) that is responsive to the biological parameter.

Certain embodiments of the present disclosure provide a method of determining the ovulation status of a subject, the method comprising using a sensor as described herein to determine the ovulation status of the subject.

Certain embodiments of the present disclosure provide a method of determining the ovulation status of a subject, the method comprising implanting a sensor as described herein into a subject and using the implanted sensor to determine the ovulation status of the subject.

Certain embodiments of the present disclosure provide a method of determining the ovulation status of a subject, the method comprising implanting a sensor as described herein under the skin of a subject and using the implanted sensor to determine the ovulation status of the subject.

Certain embodiments of the present disclosure provide a method of determining the ovulation status of a subject, the method comprising using a sensor to detect a biological parameter indicative of ovulation status, wherein the sensor comprises an optical property between 400 and 1200 nm (or about this range) that is responsive to the biological parameter.

Certain embodiments of the present disclosure provide a method of determining the ovulation status of a subject, the method comprising using a sensor comprising one or more porous silicon layers to detect a biological parameter indicative of ovulation status, wherein the sensor comprises an optical reflectance property between 400 and 1200 nm (or about this range) that is responsive to the biological parameter.

Certain embodiments of the present disclosure provide a method of determining the ovulation status of a subject, the method comprising using a sensor comprising one or more porous silicon layers to determine the ovulation status of the subject, wherein the sensor comprises an optical reflectance property between 400 and 1200 nm (or about this range) that is responsive to a biological parameter indicative of the ovulation status of the subject.

In certain embodiments, the biological parameter is detected in vivo. In certain embodiments, the biological parameter is detected in a biological fluid and/or a processed form thereof.

Certain embodiments of the present disclosure provide a method of determining the pregnancy status of a subject, the method comprising implanting a sensor as described herein into a subject and using the implanted sensor to determine the pregnancy status of the subject.

Certain embodiments of the present disclosure provide a method of identifying a subject, the method comprising using a sensor as described herein to tag the subject and thereby identify the subject.

Certain embodiments of the present disclosure provide a sensor comprising an analyte binding molecule. Sensors are as described herein. Analyte binding molecules are as described herein.

Certain embodiments of the present disclosure provide a sensor comprising one or more porous silicon layers and an analyte binding molecule.

Certain embodiments of the present disclosure provide a sensor comprising:
(i) one or more porous silicon layers, wherein the one or more silicon layers provide an optical reflectance property between 400 and 1200 nm; and
(ii) an analyte binding molecule.

Optical reflectance properties are as described herein. In certain embodiments, the optical reflectance property comprises an optical reflectance property between 600 nm and 800 nm, or about this range. Other ranges are as described herein.

In certain embodiments, the optical reflectance property changes upon binding of an analyte to the analyte binding molecule.

Analytes are as described herein. In certain embodiments, the analyte comprises one or more of a hormone, a growth factor, an antibody, an enzyme, a drug, a protein, a ligand, a nucleic acid, a small molecule, a metabolite, a cofactor, an amino acid, a vitamin, a lipid, a carbohydrate, a sugar, a cell and/or a component thereof, an inflammatory marker, a toxin, a pesticide, a metal ion, a pathogen, a bacteria, a virus, an antigen, insulin, and an antibiotic.

In certain embodiments, the analyte binding molecule is covalently linked to the one or more porous silicon layers.

Analyte binding molecules are as described herein. In certain embodiments, the analyte binding molecule comprises one or more of an antibody and/or an antigen binding fragment thereof, a receptor, a ligand, an aptamer, a nucleic acid, a protein, a small molecule, a drug, a co-factor, a virus and/or a part thereof, a carbohydrate, and a lipid.

In certain embodiments, the analyte binding molecule comprises a molecule that binds a hormone whose level is indicative of pregnancy. In certain embodiments, the analyte binding molecule comprises an antibody to luteinizing hormone and/or an antigen binding fragment thereof.

In certain embodiments, the analyte binding molecule comprises a molecule that binds a hormone whose level is indicative of ovulation status. Examples are as described herein.

Sensors comprising one or more porous silicon layers are as described herein.

In certain embodiments, the sensor comprises a single porous silicon layer. In certain embodiments, the sensor comprises a plurality of porous silicon layers. In certain embodiments, the plurality of porous silicon layers comprise a Bragg reflector. In certain embodiments, the plurality of porous silicon layers comprise a rugate filter.

In certain embodiments, the sensor comprises microcavities between porous silicon layers.

In certain embodiments, the porous silicon layers are electro-chemically etched into the silicon layer by a process utilising a composite-current time waveform.

In certain embodiments, the sensor comprises a distinguishable optical reflectance property.

In certain embodiments, the sensor comprises a film or membrane comprising the one or more silicon layers.

In certain embodiments, the sensor comprises particles comprising the one or more silicon layers.

In certain embodiments, the sensor comprises an optical fibre.

In certain embodiments, the sensor is substantially biocompatible.

In certain embodiments, the sensor comprises an in vivo half life of 2 weeks to 4 months, or about this range.

Certain embodiments of the present disclosure provide a method of detecting an analyte using a sensor as described herein.

Certain embodiments of the present disclosure provide a method of determining the ovulation status of a subject, the method comprising using a sensor as described herein to determine the ovulation status of the subject

Certain embodiments of the present disclosure provide a method of determining the pregnancy status of a subject, the method comprising using a sensor as described herein to determine the pregnancy status of the subject.

Certain embodiments of the present disclosure provide a method of identifying a subject, the method comprising using a sensor as described to tag the subject and thereby identify the subject.

Finally, standard techniques may be used for recombinant DNA technology, protein chemistry, antibody use and production, oligonucleotide synthesis, and tissue culture. Enzymatic reactions and purification techniques may be performed according to manufacturer's specifications or as commonly accomplished in the art or as described herein. The foregoing techniques and procedures may be generally performed according to conventional methods known in the art and as described in various general and/or more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), herein incorporated by reference.

The present disclosure is further described by the following examples. It is to be understood that the following description is for the purpose of describing particular embodiments only and is not intended to be limiting with respect to the above description.

### EXAMPLE 1

### Production of Implantable Biosensors

Introduction

Porous silicon (pSi) is a nanostructured material which has unique optical, electronic and biomaterials properties. The properties of pSi such as its large surface area (up to 800 m²/g), its fast preparation and its diverse and tuneable optical and surface-chemical properties make this material suitable for biosensor applications. pSi films of several micrometer thickness formed on crystalline Si by electrochemical etching are subject to thin film interferences when illuminated with white light. These can be detected with a CCD spectrometer as Fabry-Perot fringes which contain information about the refractive index of the porous layer (n) and the thickness of the layer (d). A change in the refractive index of the porous layer (e.g. upon binding of biological macromolecules) manifests itself in a shift of the fringe pattern and a corresponding change in the effective optical thickness (EOT) of the layer (2nd). By varying the current density during the etching process, pSi can assume double- or multilayered structures. Alternating between two distinct currents in real time results in discrete modulations in porosity and hence in refractive index with depth in a stepwise fashion results. This gives rise to Bragg reflectors in which only light at a defined wavelength corresponding to the photonic bandgap is reflected. In rugate filters, which are obtained by the sinusoidal variation of the current density, sidelobes seen in Bragg reflectors are reduced and cleaner resonances are produced. However, peak reflectance is also reduced. Microcavities are 1D photonic bandgap structures that include a spacer layer positioned between two Bragg reflectors resulting in the formation of a narrow photonic resonance, which appears as a dip in the reflectance spectrum and is highly sensitive to changes in refractive index, such as those arising from binding of biomolecules in the pores.

pSi is a remarkably inert and non-inflammatory material within the body. The main advantage over other biomaterials lies in its ability to degrade completely in aqueous solutions into non-toxic silicic acid, the major bioavailable form of silicon in the human body. The structural integrity of the material can be preserved and the degradation kinetics controlled from hours to months by modifying the surface using silanisation, hydrosilylation and thermal carbonisation.

In this work, we investigate various pSi reflectors for their ability to produce reflectance signals when placed underneath cadaver skin. This study provides fundamental understanding that is required for the design of implantable optical biosensors.

Experimental Details

Fabrication of pSi Films

pSi thin films with single layers, rugate filters and microcavities configurations were fabricated using a computer controlled Keithley 2425 source meter. Samples were prepared by electrochemical anodization of p-type boron doped crystalline silicon (c-Si) wafers (5 mΩm, resistivity and <100> orientation), in a 1 HF:2 EtOH (v/v) solution, at room temperature. For each film, the initial pSi parasitical layer was removed using a 1 M solution of sodium hydroxide (as described in H. Ouyang, M. Christophersen, R. Viard, B. L. Miller, and P. M. Fauchet. Macroporous silicon microcavities for macromolecule detection. Advanced Functional Materials, 15(11):1851, 2005. DOI:10.1002/adfm.200500218), and then the etching of the final layers was carried out.

Current densities between 90 to 160 mA cm⁻², for etching times from 200 to 700 s, were used to fabricate pSi single layers. In order to characterize pSi single layers, the absolute reflectance of each fabricated sample was measured and fitted using conventional algorithms, transfer matrix method and the effective medium theory proposed by Looyenga (as described in L. N. Acquaroli. Propiedades ópticas de silicio poroso nanoestructurado. Tesis Doctoral, 2011. Argentina.) From this fitting procedure, porosity and thickness of each pSi film were obtained. In order to prepare pSi microcavities, two different current densities were chosen with their respective etching rates and porosities, characterized from pSi single layers method. Then, quarter wavelength layers (as described in A. V. Kavokin, J. J. Baumberg, G. Malpuech, and F. P. Laussy. Microcavities. Oxford University Press, New York, 1st. Edition, 2007) were designed, and fabricated at different wavelengths in order to obtain different optical responses for the microcavities. pSi-based rugate filters were fabricated following using the methodology as described in E. Lorenzo, C. J. Oton, N. E. Capuj, M. Ghulinyan, D. Navarro-Urrios, Z. Gaburro, and L. Pavesi. Porous silicon-based rugate filters. Applied Optics, 44(26):5415, 2005. DOI:10.1364/AO.44.005415, with an average current density 85 mA cm⁻² and etching times values of 340 and 450 s.

For improved chemical stability in aqueous environment, freshly etched pSi films were oxidized during 1 h under ozone ambient, at room temperature, employing and ozone generator, for example as described in L. N. Acquaroli, R. Urteaga, and R. R. Koropecki. Innovative design for optical porous silicon gas sensor. Sensors and Actuators B: Chemical, 149(1):189, 2010. DOI:10.1016/j.snb.2010. 05.065.

Preparation of Cadaver Skins

Rabbit and guinea pig skins provided by the Large Animal Research and Imaging Facility (Adelaide, Australia) were utilized in the experiment. Skins samples were stored in saline solution inside a fridge at 4° C., during the whole experiments. The hair and the fat layer of the skins were removed with the help of a metal scalpel before optical measurements, so as to reduce the scattering and absorption of light. The thicknesses of the skins were measured with a micrometer, and their values vary around 1±0.05 mm for the guinea pig skin, and 2±0.1 mm for the rabbit skin. Glycerol at 87% and a saturated sucrose solution were used as optical clearing agents for different skins samples. Skins were incubated in these two solutions at room temperature throughout experiments.

Reflectance Measurements through Skin

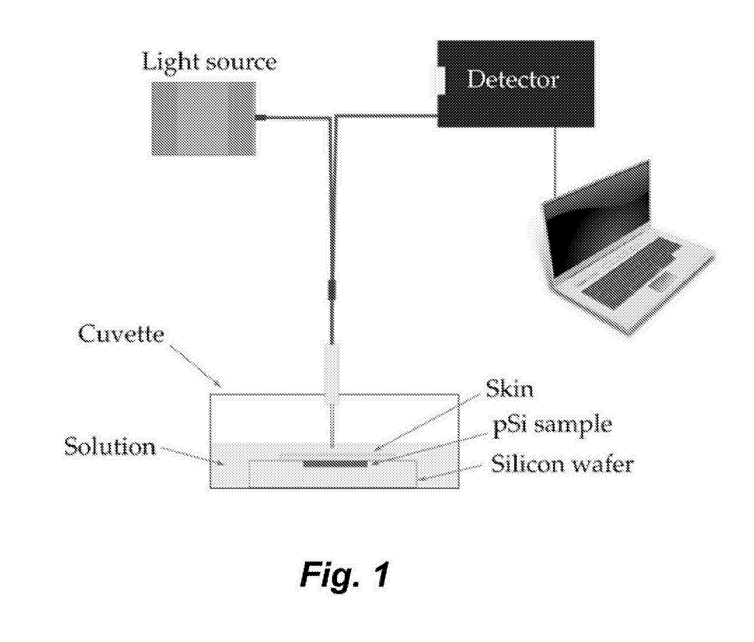

Optical reflectance measurements through different animal skins were carried out using a LS-1 tungsten halogen light source and an USB4000 Ocean Optics spectrometer in the visible range. A scheme of the experimental setup is shown in FIG. 1, where the skin is placed on the top surface of the pSi thin film. Reflection spectra through skins were taken in two different solutions, glycerol and sucrose.

(ii) Results and Discussion

Since cadaver skin rapidly turns opaque, optical measurements of skin-embedded pSi reflectors were performed in glycerol and sucrose solution. These solutions reduce scattering and absorption of animal and human skin, avoid dehydration and provide refractive index matching.

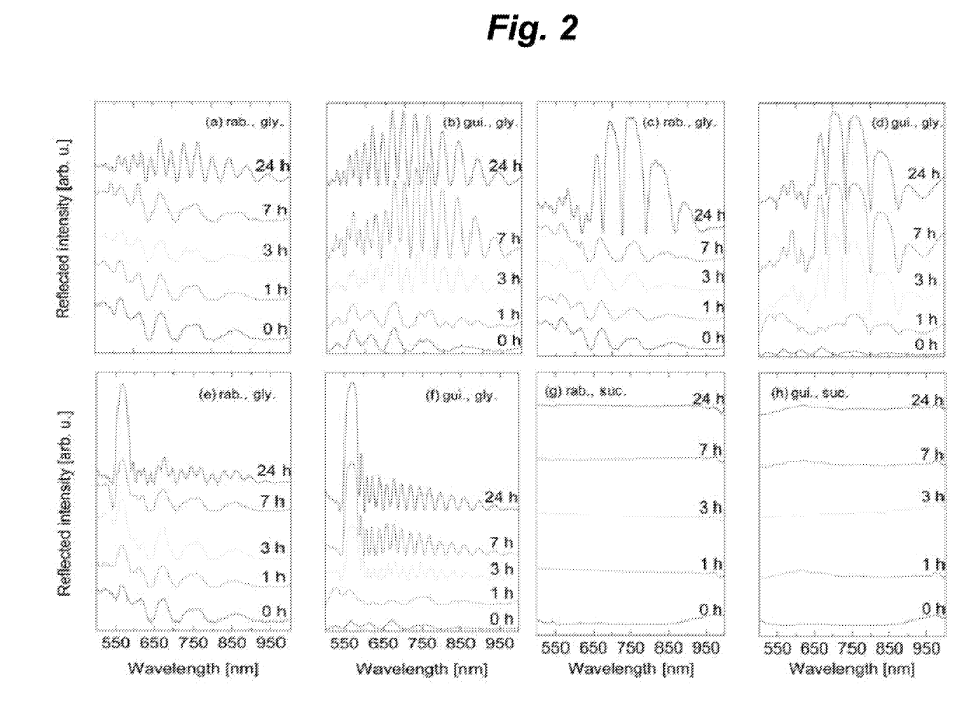

Reflection spectra for pSi single layer films, microcavities and rugate filters placed underneath guinea pig and rabbit skin are presented in FIG. 2. The time evolution of the reflection spectra for distinct pSi thin films is shown in each graph after immersion in glycerol (FIG. 2a-f). Spectra in FIGS. 2a, 2c, 2e and 2g correspond to pSi samples placed underneath rabbit skin, whereas FIGS. 2b, 2d, 2f and 2h show spectra from pSi reflectors underneath guinea pig skin. From FIG. 2, it is possible to observe that cadaver skin scattering can be overcome by immersion of the reflector-skin assembly in either glycerol or sucrose solution. For rabbit skin, 24 h treatment was required to obtain high-quality reflectance spectra whereas for guinea pig skin, 3 h immersion in glycerol was sufficient. One simple explanation is rabbit skin is thicker than that of guinea pig and that the index matching solution needs more time to take effect. Alternatively, glycerol might interact differently on the two animal skin types due to the different texture and composition, and this also could lead to the observed discrepancy.

Measurements of reflection with skin immersed in sucrose solution were also performed. Unfortunately, no optical signal could be obtained after many hours of immersion of the reflector-skin assemblies (FIG. 2g-h). This was surprising since sucrose has been used successfully for the purpose of refractive index matching of cadaver skin in the literature.

According to these results, and especially those for guinea pig skin in glycerol solution, optical signals from pSi reflectors can be readily read out through the skins, and processed either by Fourier transforming single layers spectra, or by following the peak of the narrow filter-like spectral features of multilayer pSi structures. Although animal skin was used in this work, glycerol has been previously used as an index matching fluid for human skin. Our results therefore set the stage for the development of pSi based implantable biosensors which take advantage of the optical properties and the good biocompatibility and biodegradability of pSi. Furthermore, pSi reflectors can be fabricated in the form of membranes or microparticles which can be easily implanted or even injected underneath the skin. If the readout occurs through an optical fiber, a geometric sample area of less than 0.5 mm² is required.

The reflection spectra taken in the above experiments cover the wavelength range from 500 nm to 1000 nm, in agreement to the so-called optical window in biological tissue. By controlling the fabrication conditions for pSi, it is possible to tune the optical response within this window. Our results suggest that pSi based implantable biosensors could be implemented over a broad wavelength range, potentially allowing multiplexing of implantable biosensors.

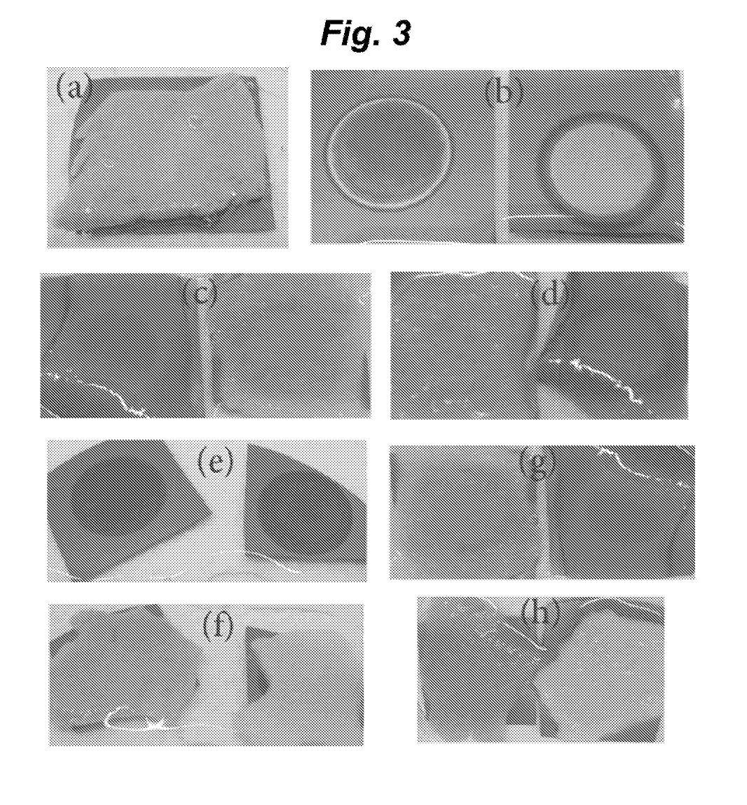

In FIG. 3, pictures of pSi thin films are shown either covered with rabbit skin and guinea pig skins using either glycerol of sucrose for index matching. FIG. 3a presents a pSi reflector underneath hairless guinea pig skin at time zero of incubation in glycerol. At this point, the skin is so opaque that it is difficult to see the pSi reflector underneath. FIG. 3b shows two different pSi thin films in glycerol solution. In FIGS. 3c and 3d, the same pSi samples covered by rabbit and guinea pig skins after 24 h of incubation in glycerol are shown. The characteristic colors of pSi thin films are clearly visible through the skin, in agreement to reflection spectra shown in FIG. 2.

On the other hand, in FIGS. 3e and 3f, it can be seen pSi samples in sucrose solution, without and with rabbit and guinea pig skins after 24 h treatment on top, respectively. Again, sucrose solution did not have the desired effect on skin transparency and the pSi reflectors can hardly be discerned underneath the skin samples.

FIGS. 3g and 3h depict the transition from skin samples incubated in glycerol for 24 h to the same skin samples after incubation in PBS solution. In FIG. 3g, the treated skin is almost transparent after incubation in glycerol for 24 h. Oppositely, after incubation in PBS the skin remained opaque in consistence with other studies.

Further proof that pSi reflectors underneath skin samples can be optically interrogated was obtained by taking the Fourier transforms of the reflection spectra of the single layer pSi film underneath a skin sample shown in FIG. 2b. The Fourier transform gave the optical thickness of the pSi film which was compared to the optical thickness of a freshly etched pSi sample determined by fitting the absolute reflectance spectrum. The value obtained for the optical path of the as prepared pSi film was 7200 nm, whereas that of the pSi sample underneath the skin was 7370 nm. These results are in good agreement since they were taken from different methods. Differences in the exact values are consequence of the fabrication method and can be explained in terms of a small radial variations in pSi film thickness.

Having shown the ability to obtain optical signals from pSi films through animal skin, we next step determined the most suitable wavelength band for this work. In order to optimize the wavelength, four pSi microcavities showing resonances at different wavelengths across the optical window were prepared and tested underneath skin samples in glycerol. Although animal skin seemed to be transparent within the entire optical window, optimization of the wavelength range with different pSi multilayers were carried out taking into account the possible effect of glycerol considering a system as that one presented in FIG. 1. pSi microcavities were chosen among the different pSi architectures, due to the fact that they are more suitable to detect very small changes of refractive indexes than rugate filters and Bragg mirrors, by using the sharp and narrow resonances in their optical spectra.

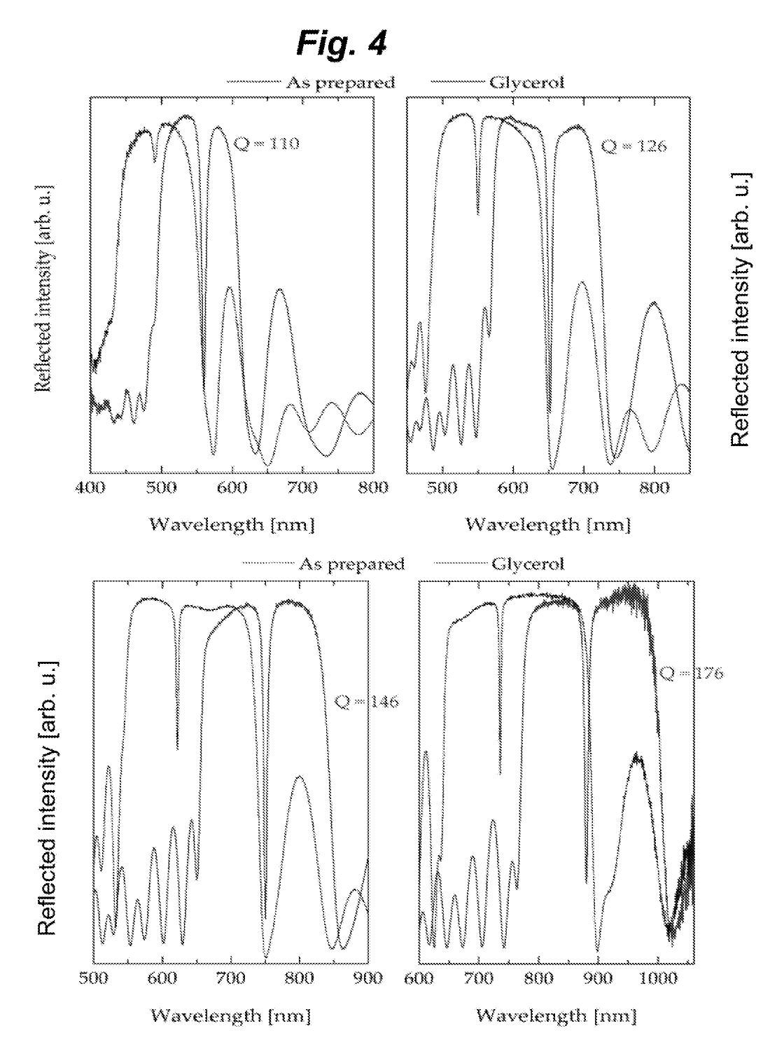

FIG. 4 shows the reflection spectra of four pSi microcavities centered at four different positions across the optical window. For each microcavity, the quality factor (Q, simply estimated as the central wavelength divided by the full width at half maximum of the resonance—A. V. Kavokin, J. J. Baumberg, G. Malpuech, and F. P. Laussy. Microcavities. Oxford University Press, New York, 1st. Edition, 2007) was calculated. As seen from FIG. 4, the Q factor increased concomitant to the central wavelength of the resonance, with values 110, 126, 146 and 176, respectively. It is also observed that owing to the high absorption of silicon in the short wavelength region, microcavities present a better shape when they are centered at higher wavelengths.

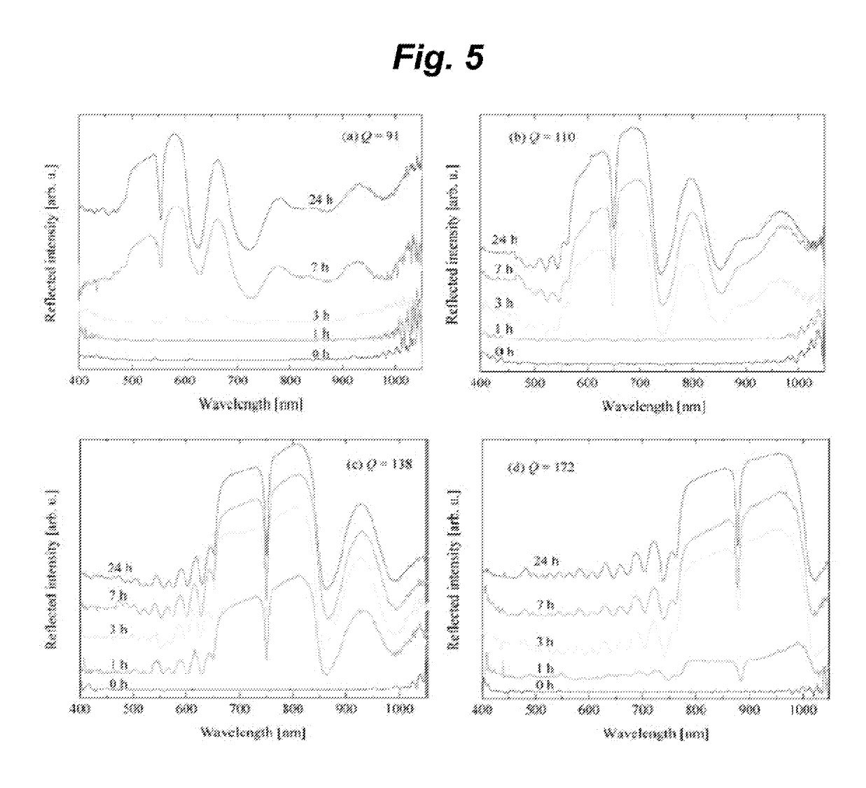

After the microcavities were characterized, they were placed under guinea pig skin and the evolution of the reflection spectra of these samples for different incubation times in glycerol was studied. Results are presented in FIG. 5. The results confirm the earlier results in terms of 3 hours being sufficient to obtain transparency but improvements in signals were achieved up to 24 hours.

In order to determine which microcavity was best suited for implanted biosensor applications, the Q factors of four pSi microcavities were assessed and compared. It can be seen from the data that the best Q factors came from microcavities centered at higher wavelengths. Since the Q factor measures how far a microcavity is from the ideal condition due to light absorption and other dissipation mechanisms (for example as described in A. V. Kavokin, J. J. Baumberg, G. Malpuech, and F. P. Laussy. Microcavities. Oxford University Press, New York, 1st. Edition, 2007), these results indicate that skin (after treatment with glycerol for 24 h) is transparent over the analyzed wavelength range, and that the optical quality of the microcavity is mainly subject to the absorption spectrum of silicon. Also, the Q factor value for the spectrum of FIG. 5d remained almost the same with and without skin. This can also be explained taking into account the low absorption and scattering mechanisms at longer wavelengths within the spectral range considered. However, if a particular sensing application requires a different working wavelength, shorter wavelength can be used, as shown in FIGS. 5b and 5c, where a good quality factor still remains compared to those reflectors without skin (FIG. 4).

This set of experiments provide design principles for pSi based optical biosensors implanted under the skin. Although glycerol-treated animal cadaver skin is different from live animal skin, both have similar optical properties. Refractive index matching fluids such as glycerol have been shown to be compatible with living tissue and may possibly enhance in vivo deep-tissue imaging.

Conclusions

We have explored the use of interferometry on pSi thin films to detect optical signals through animal cadaver skin. An assessment of the optical response of pSi thin films suggests that the addition of refractive index matching solutions on cadaver skin mimics the conditions of live animal skin and allows the acquisition of reflectance spectra. It was demonstrated that prolonged incubation of tissues in glycerol improved the quality of the reflectance spectra through both rabbit and guinea pig skin, with the latter skin type affording higher quality spectra. The work described here may be used in the development of pSi biosensors implanted underneath the skin in the form of films, membranes or particles and read through the skin using an external optical probe. This technology has wide-spread applications for the management of human diseases and for detection of physiological changes in farmed animals.

### EXAMPLE 2

### Preparation of Sensors Using an Antibody to a Hormone

Porous silicon was prepared as described above.

If porous silicon particles are required, electrochemical etching of crystalline silicon may be performed, followed by lifting up the porous membrane, fracturing the membrane into particles by ultrasonication and sieving to obtain the desired size distribution. Optical and electro microscopy can be used to determine the size distribution. Nitrogen sorption analysis can be used to calculate the average pore channel diameter, the porous volume, and the specific surface area of the nanoparticles.

Immobilization of antibodies on the porous silicon surface may be undertaken by coupling between the porous silicon and the antibody using a semicabazide functional linker. Silicon hydride terminated porous silicon is first hydrosilylated with tert-butyl-2-[(allylamino)carbonyl]hydrazine-carboxylate, a Boc-protected semicarbazide functional alkene. Protection of the semicarbazide is required to avoid interference with the hydrosilylation reaction.

In parallel, chemical modifications may be performed on an antibody of interest to prepare them for coupling with the semicarbazide-functionalized pSi nanoparticles. The carbohydrate side-chain on the Fc fragment of the antibodies may be first oxidized using NaIO₄ to generate aldehyde functional groups, as described in Wolfe, C. A. C. and Hage, D. S., (1995) Analytical Biochemistry 231, 123-130. These react readily with the hydrazine functionality of the deprotected semicarbazide and will form a very stable covalent bond. Carbodiimide coupling chemistry may also be used. A short oxidation procedure assists in retaining antibody activity and typically oxidation times of 30 minutes or less and rapid removal of the oxidant during the workup are to be used. Gel filtration purification process may be used purify, or alternatively, successive dialyses can be used. Antibodies are then coupled to the semicarbazide-functionalized porous silicon.

Monitoring of analyte binding may be performed quantitatively with sub-nanometer resolution using reflectance spectroscopy or qualitatively using the unaided eye. Quantitative reflectance signals may be processed either by Fourier transforming single layers spectra, or by following the peak of the narrow filter-like spectral features of multilayer pSi structures. The analyte binding will lead to red shifts in the effective optical thickness (EOT) or in the reflectance peak corresponding to several nanometers and correlating with the concentration of the analyte.

### EXAMPLE 3

### Introduction of a Sensor into an Animal

In one embodiment, the sensor is in the form of a disk of 0.5 cm² diameter and may be implanted subcutaneously or injected subcutaneously in the form of microparticles through a needle. Reflectance signals may be monitored through the skin using a quantitative fibre optics reflectance spectrometer or, qualitatively, by the unaided eye.

Alternatively, particles may be injected subcutaneously with an applicator and a scanner (eg a hand held scanner) moved over the area to detect optical signatures.

### EXAMPLE 4

### Porous Silicon Biosensor—Fabrication and Characterisation Results

Porous silicon was fabricated as follows: Porous silicon was prepared from p-type, boron doped crystalline (100) silicon wafers of 0.001-0.0005 Ωcm resistivity. Silicon pieces of ˜1.5 cm² were assembled into a teflon etching cell, with a platinum electrode cathode and an aluminium tape backing anode. An ethanolic solution of HF (3:1 HF_{(48%)aq}:EtOH for pSi rugate filters) was used for etching and each silicon piece was cleaned with EtOH and acetone prior to etching. An initial etch at 50 mA.cm⁻² for 30 sec in 3:1 HF_{(48%)aq}:EtOH, followed by dissolving the porous layer in 0.5 M NaOH and washing in MilliQ H₂O and EtOH was performed. This was to remove the parasitic layer that can inhibit adequate pore formation in the subsequent etching steps. pSi rugate filters were fabricated by applying a sinusoidal current, cycled between 40 and 100 mA.cm⁻², with a period of 8.5 sec for 25 cycles.

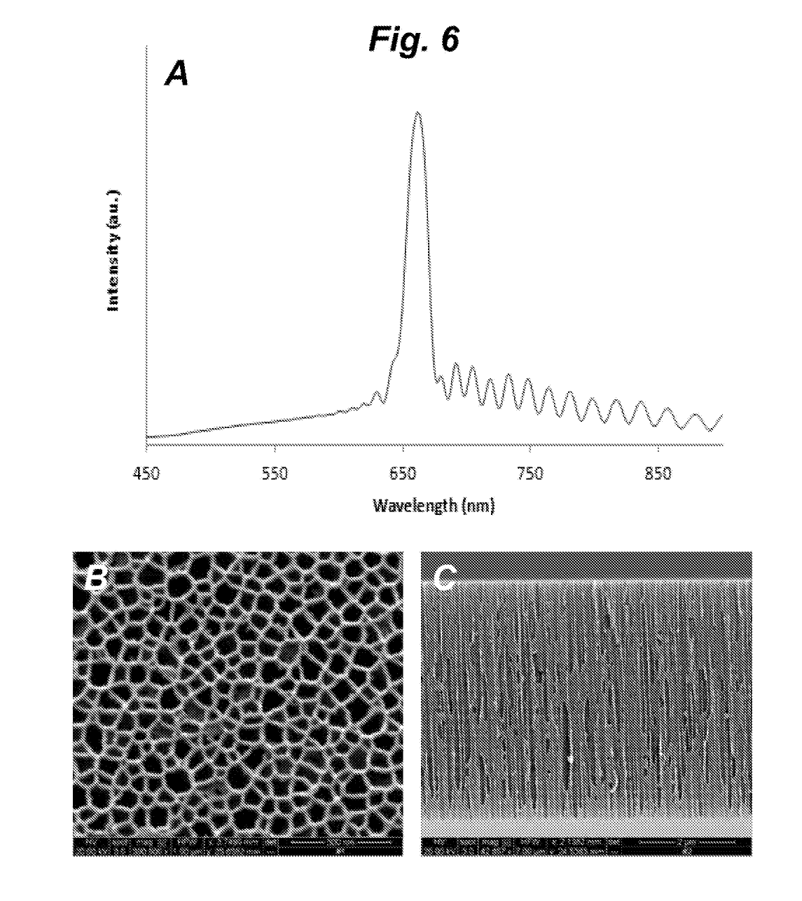

FIG. 6A shows the white light reflectivity spectrum of porous silicon rugate film in aqueous medium. As can be seen, the optical reflectance spectra display a single, sharp reflectance peak. The position (max wavelength) of this peak is influenced by the material within the pores. FIGS. 6B and 6C show SEM images of porous silicon film. a) top view, scale bar 300 nm and b) cross section, scale bar 2 mm.

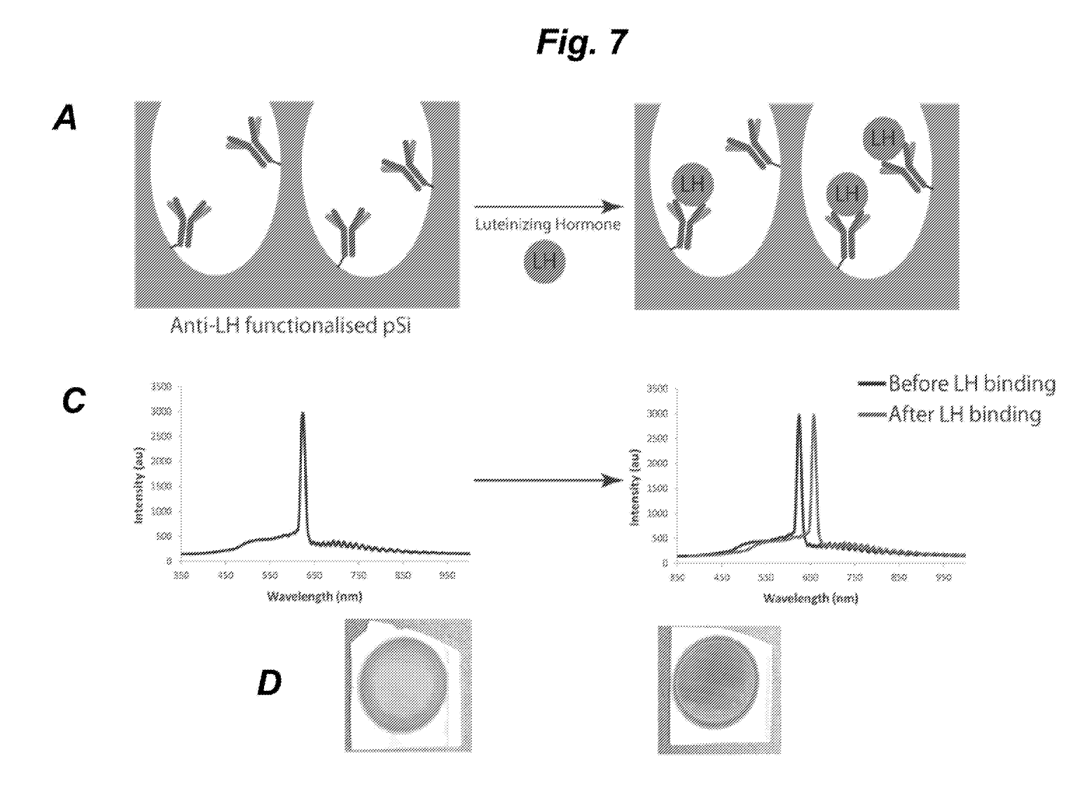

FIG. 7 shows a schematic for the detection of LH using a porous silicon biosensor. Panel A shows a schematic of porous silicon functional with anti-LH antibody, and panel B shows binding of LH to the antibody. Panel C shows the expectation that the detection of LH binding to the antiLH within the pores will be determined by the ‘red’ shift of the pSi photonic peak. Panel D shows two representative photos of porous silicon films, one green and one red, to display the observable difference between surfaces.

### EXAMPLE 5

### Porous Silicon Surface Modification

Modification by Thermal Hydrocarbonization (Method 1)

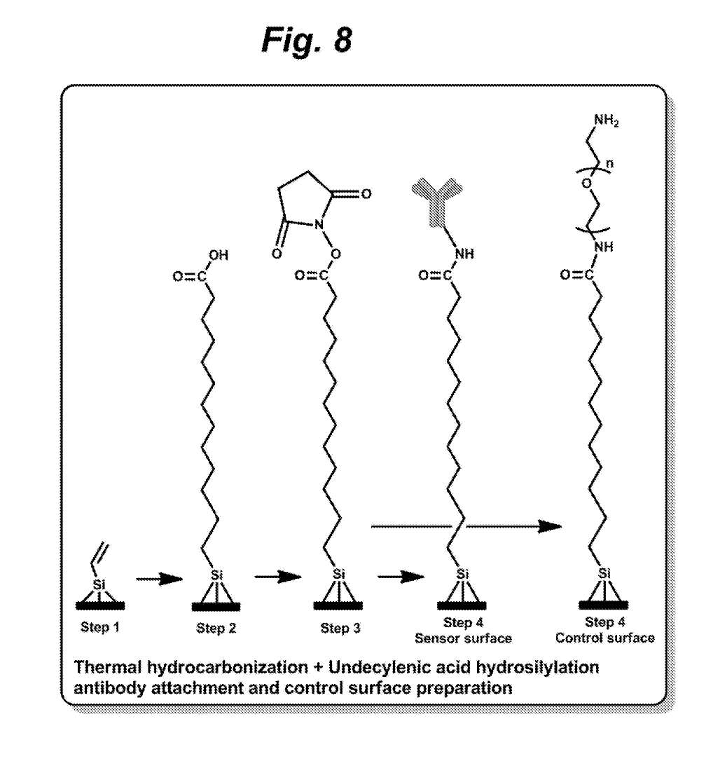

The method of porous silicon surface modification by thermal hydrocarbonization is shown in FIG. 8. This methodology involves pSi surfaces modified to improve stability and to allow antibody conjugation. The methodology involves the following steps:

Step 1: thermal hydrocarbonization: Freshly etched pSi photonic crystals were placed into a sealed glass tube and purged with N_{2(g)} for a period of 45 min. Acetylene gas was then introduced into the tube at a ratio of 1:1 acetylene:N_{2(g)} for a period of 15 min. Under the flow of acetylene and N_{2(g)}, the tube was then placed into a furnace, maintained at 500° C. for a further 15 min. The acetylene supply was cut 30 sec before the final 15 min was complete, after which the tube was removed from the furnace and allowed to cool to room temperature under N_{2(g)} flow.

Step 2: thermal hydrosilylation with undecylenic acid: Thermally hydrocarbonized pSi photonic crystals were placed in a 0.1 M solution of undecylenic acid in mesitylene. The solution was heated for 15 hr at 140° C. after which the sample was allowed to cool and washed with acetone and ethanol before being dried under a flow of nitrogen.

Step 3: conversion of terminal carboxylic acid into amine reactive n-hydroxysuccinimide ester: pSi photonic crystals functionalised with undecylenic acid were activated by reaction with 10 mM EDC/NHS in PBS (pH 7.4) for 1 hr, after which they were rinsed thoroughly with MilliQ H₂O.

Step 4 (sensor surface): luteinizing hormone antibody conjugation: A 15 μg/mL solution of LH antibody was immediately allowed to react with the surface for 15 hr at room temperature. After the antibody conjugation the surface was washed three times with PBS and finally stored in fresh PBS before use in in vivo experiments.

Step 4 (control surface): conjugation of amino-polyethylene glycol: A 1 mM solution of bisamino polyethyene glycol was immediately allowed to react with the NHS activated surface for 15 hr at room temperature. After the antibody conjugation the surface was washed three times with PBS

Modification by Thermal Carbonization (Method 2)

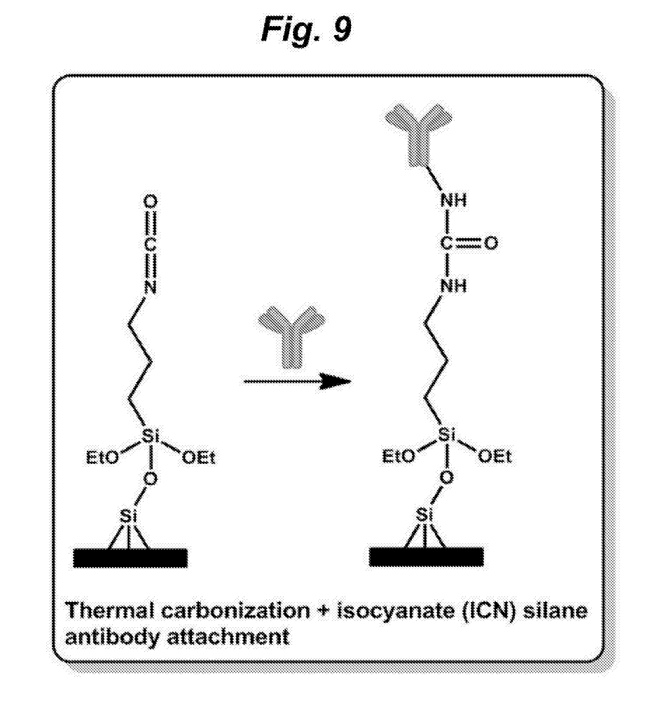

The method of porous silicon surface modification by a modified thermal hydrocarbonization is shown in FIG. 9. This methodology involves the following steps:

Step 1: thermal carbonization (not shown): Freshly etched pSi photonic crystals were firstly thermally hydrocarbonized (as above) then placed into a sealed glass tube and purged with N_{2(g)} for a period of 45 min. Acetylene gas was then introduced into the tube at a ratio of 1:1 acetylene:N_{2(g)} for a period of 10 min. The acetylene flow was stopped at 9 min 30 sec and at 10 min the tube was then placed into a furnace, maintained at 800° C. for a further 10 min. The tube was then removed from the furnace and allowed to cool to room temperature under N_{2(g)} flow.

Step 2: HF treatment (not shown): Thermally carbonized porous silicon were soaked in 1:2 HF:EtOH for 5 min then washed with copious amounts of ethanol and dried under N_{2(g)}.

Step 3: conjugation of isocyanatopropyl triethoxy silane (ICN): Porous silicon surface was reacted for 10 min in a 50 mM solution of ICN in 5 mL anhydrous toluene. The surface was washed with toluene and dried under N_{2(g)}.

Step 4: conjugation of antibody: A 15 μg/mL solution of antibody was immediately allowed to react with the surface for 3 hr at room temperature. After the antibody conjugation the surface was washed three times with PBS and finally stored in fresh PBS before use in in vivo experiments.

Modification by Thermal Carbonization (Method 3)

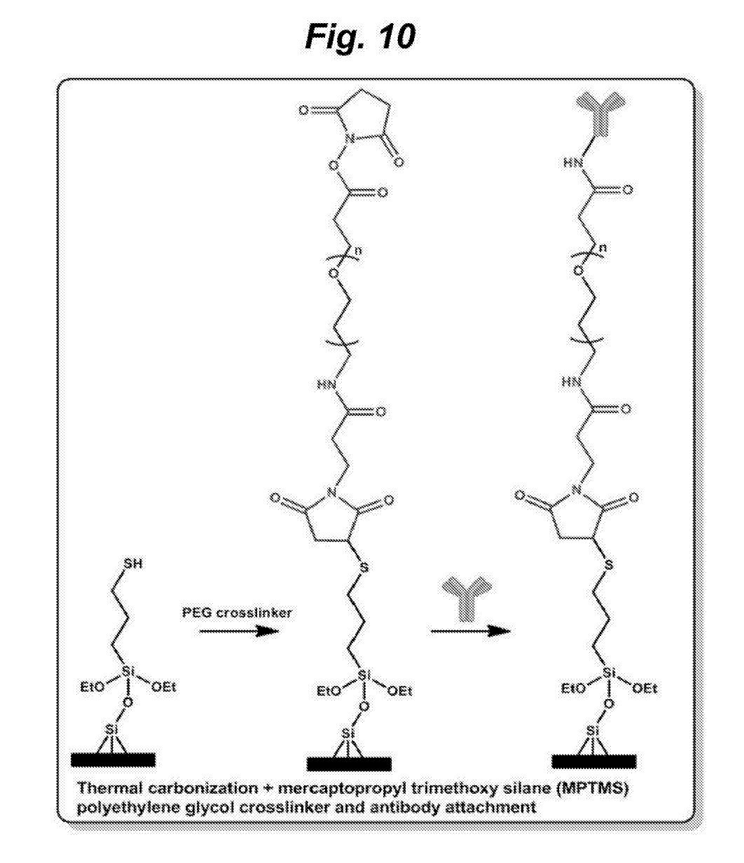

The method of porous silicon surface modification by another modified thermal hydrocarbonization is shown in FIG. 10. This methodology involves the following steps:

Step 1: thermal carbonization (not shown): as above

Step 2: HF treatment (not shown): as above

Step 3: conjugation of mercaptopropyl trimethoxy silane (MPTMS): as above, using MPTMS in place of ICN

Step 4, conjugation of polyethylene glycol (PEG) crosslinker: 1 mg/mL maleimide-PEG-NHS crosslinker was reacted with the MPTMS porous silicon surface for 2 hr, after which it was washed with MilliQ H₂O.

Step 5, conjugation of antibody: as above

### EXAMPLE 6

### Porous Silicon Biosensor—in vitro Results for Detection of LH

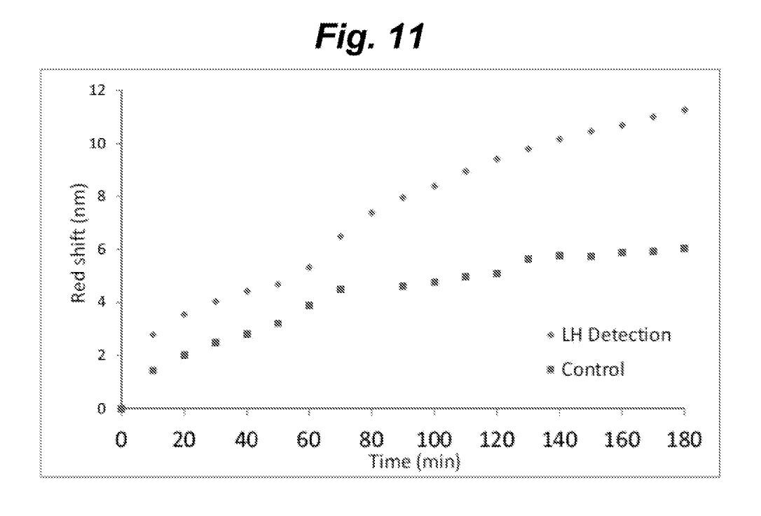

FIG. 11 shows detection and control porous silicon sensor surfaces, incubated in a solution of LH at 100 μg/mL. The peak shift indicates LH binding to the porous surface.

FIG. 11 method: An anti-LH functionalised (thermal hydrocarbonization method) pSi surface was clamped into a plexi glass flow cell and a solution of LH (100 μg/mL) in PBS was introduced across the surface. The reflectance spectra from the surface were recorded every minute for a period of 180 minutes using a bifurcated fibre optic probe connected to a tungsten light source (incident light) and an Ocean Optics USB2000 spectrometer (reflected light). The position (wavelength) of the reflection peak was recorded and plotted against time.

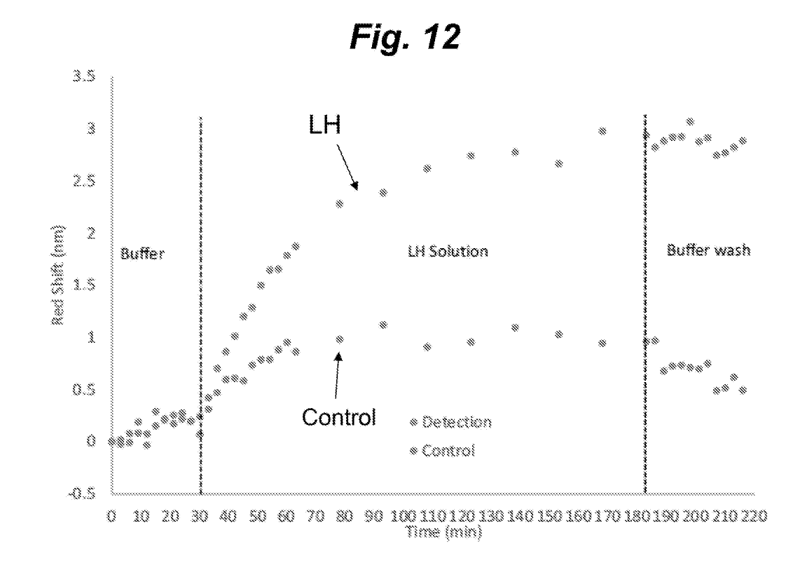

FIG. 12 shows an improved biosensor surface having a larger pore diameter, optimised antibody coverage. The figure shows detection and control porous silicon sensor surfaces, incubated in a solution of LH at 60 μg/mL. The major improvement is an increase in pore diameter, allowing easier diffusion of LH into the pores. The sensor has been improved by performing an etching process with a vertically aligned electrode.

This produces a gradient of pore sizes across the surface propagating from largest to smallest, away from the position of the electrode. The biosensor is then measured at the site of the largest pores. The etching process is the same as described above, with the only difference being the vertical alignment of the electrode. The biosensing run in this case (FIG. 12) was conducted similarly to previously. In this case however measurements were recorded in buffer for 30 min, LH (60 μg/mL) solution for 150 min and buffer again for 30 min.

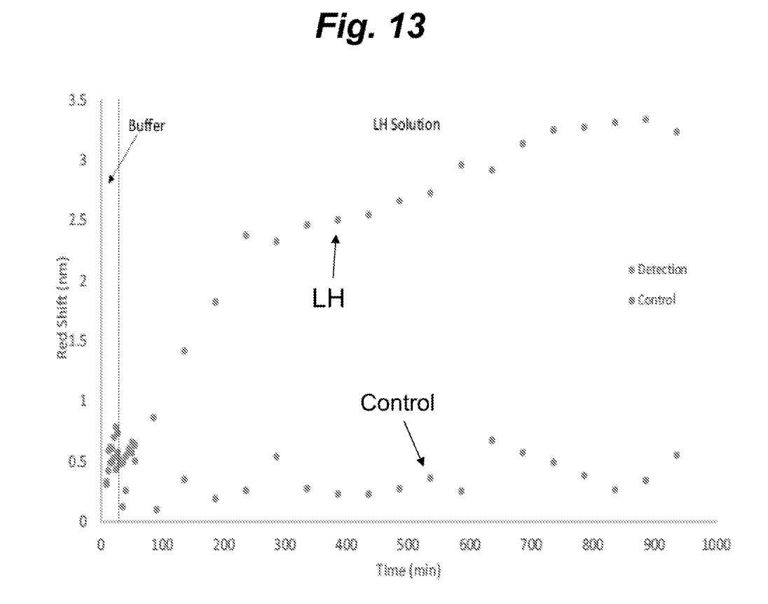

FIG. 13 shows 1 μg/mL LH in continuous flow system for 15 hr. The figure shows the detection and control porous silicon sensor surfaces, exposed to a continuous flow of LH solution at 1 μg/mL. The detection shows an accumulation of the LH on the surface over time, while there is no LH binding to the control surface. For this experiment, 8 mL of LH (1 μg/mL) solution was recirculated over the biosensor surface for a period of 15 hr. Spectra were measured once every 5 min during the LH detection timeframe.

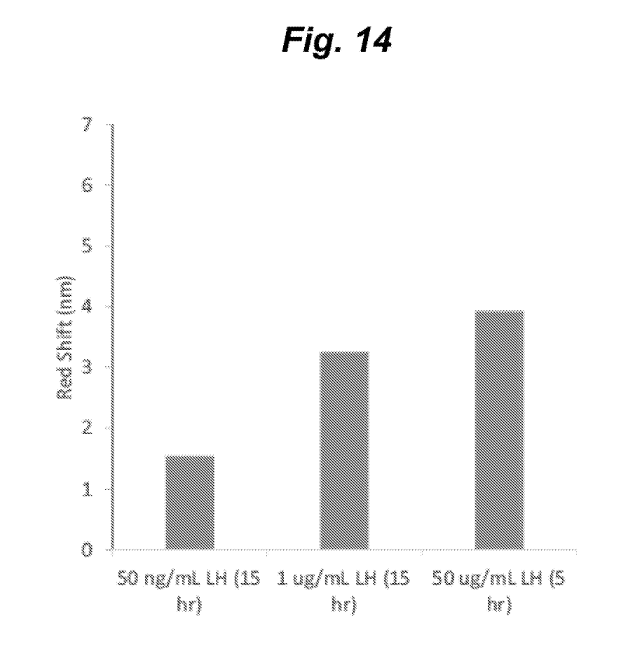

FIG. 14 shows collated data for detected red shift of sensor, at different levels of LH. The figure shows the maximum response (red shift) of the pSi biosensor surface at different LH concentrations.

### EXAMPLE 7

### Mammalian Cell Culture on Porous Silicon

To study if a conventional rugate biosensor THC treated is cytotoxic, fibroblasts were cultured on the surface (direct culture) or the cells were cultured in medium incubated with the said biosensor (indirect culture).

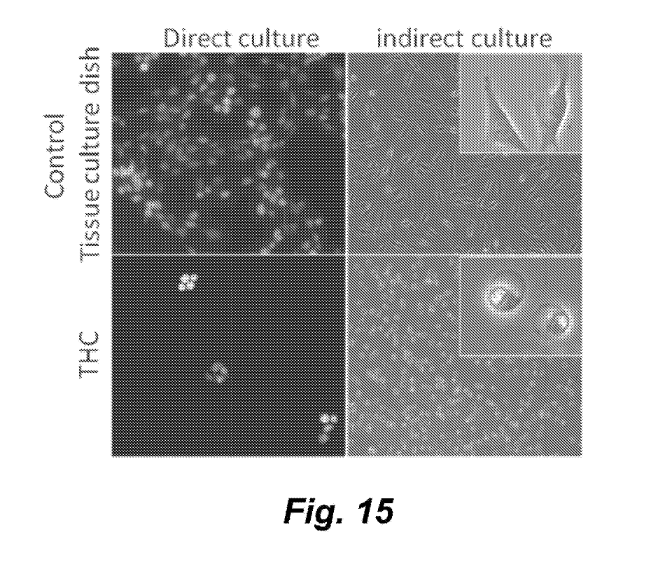

FIG. 15 shows FDA stained cells showing cell morphology on two surfaces (left panels). Cells are rounded up on THC surface. In the right panel, phase images showing cell morphologies, indicating conventional THC surfaces possesses certain degree of cytotoxicity.

### EXAMPLE 8

### Mammalian Cell Culture on Porous Silicon: Stimulation of Fibrotic Matrix

To eliminate cytotoxicity on conventional THC, an “aging” (or “pre-incubating”) process was investigated.

Fibroblasts were cultured on “aged” (or “pre-incubated”), namely PITHC, surfaces as compared to normal tissue culture surface, and THC treated flat silicon. “Aged” (or “pre-incubated”) PITHC was obtained by incubating crude THC treated pSi in physiological buffer (DMEM with full serum) at 37° C. for an optimized timeframe, such that optical performance is not impeded. Cells were then cultured on top of this surface for 24 hours, followed by staining and imaging]

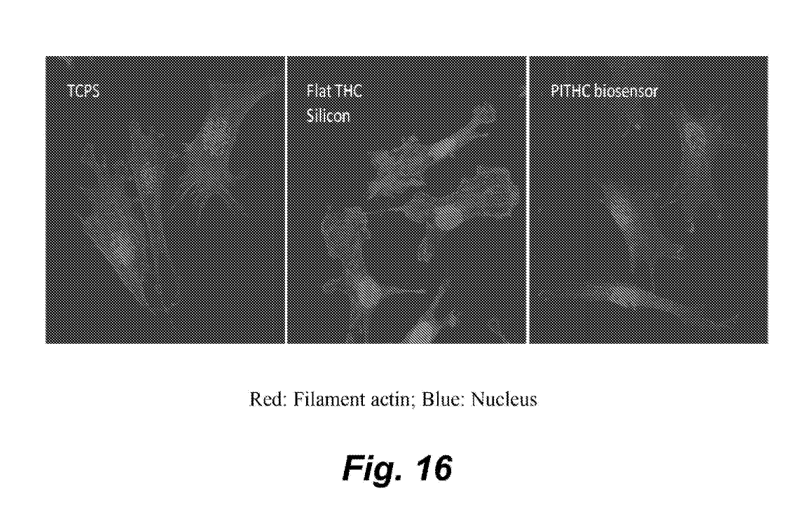

The results are provided in FIG. 16, which shows a confocal fluorescence image of fibroblasts on these surfaces. The result indicates that fibroblast could adhere and spread normally as if they are on TCPS.

To study if fibroblasts could populate on PITHC biosensor surface, and form fibrotic tissue on it, fibroblasts were cultured on porous silicon for 21 days, with and without TGFβ1 stimulation.

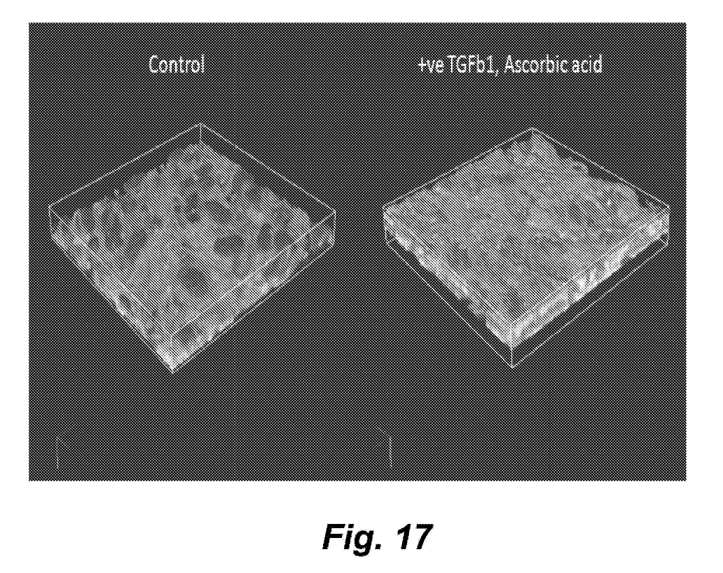

The results are provided in FIG. 17, which shows a confocal fluorescence image of fibroblasts on porous silicon with and without stimulation by TGFβ1. Firstly, result indicates that fibroblasts could populate and form normal tissue on porous silicon biosensor (PITHC). Secondly, fibroblast secreting excessive collagen 1 into its extracellular matrix under the stimulation of TGFβ1, meaning that phenotypic fibroblast function is not impeded. These indicate the cytocompatibility of the PITHC surface.

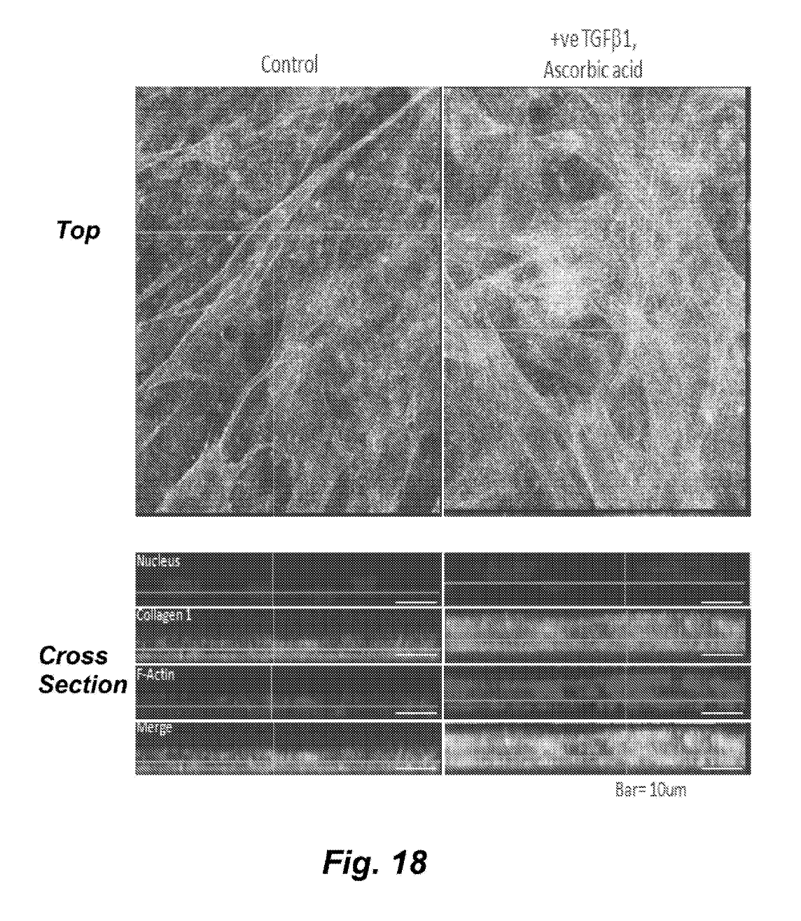

FIG. 18 shows a top view and cross section of control and TGFβ1 stimulated fibroblasts on porous silicon. The result indicates that stimulated cells form a thicker tissue, composing of more collagen 1. This more closely resembles an in vivo situation.

### EXAMPLE 9

### Mammalian Cell Culture on Porous Silicon: Analyte Detection through Fibrotic Matrix

Fibrotic tissues formation over a biosensor is often the main reason for failure, as this reduces the interaction between biosensor and analyte. To investigate whether a porous silicon rugate film covered with fibrotic tissue was still able to measure analyte concentration, fibroblasts were cultured on biosensors for 21 days, with TGFβ1 stimulation. Sucrose was then flowed across the porous silicon surface and the optical response of the rugate film was measured. The biosensor used here is the same as described in Example 5. The procedure of cell fibrotic matrix formation is the same as to the one described in Example 5. The biosensor surface with fibrotic matrix was incubated in different concentration solutions of sucrose and the reflectance spectra were recorded. The surface was incubated for 1 min in each solution before the spectrum was recorded.

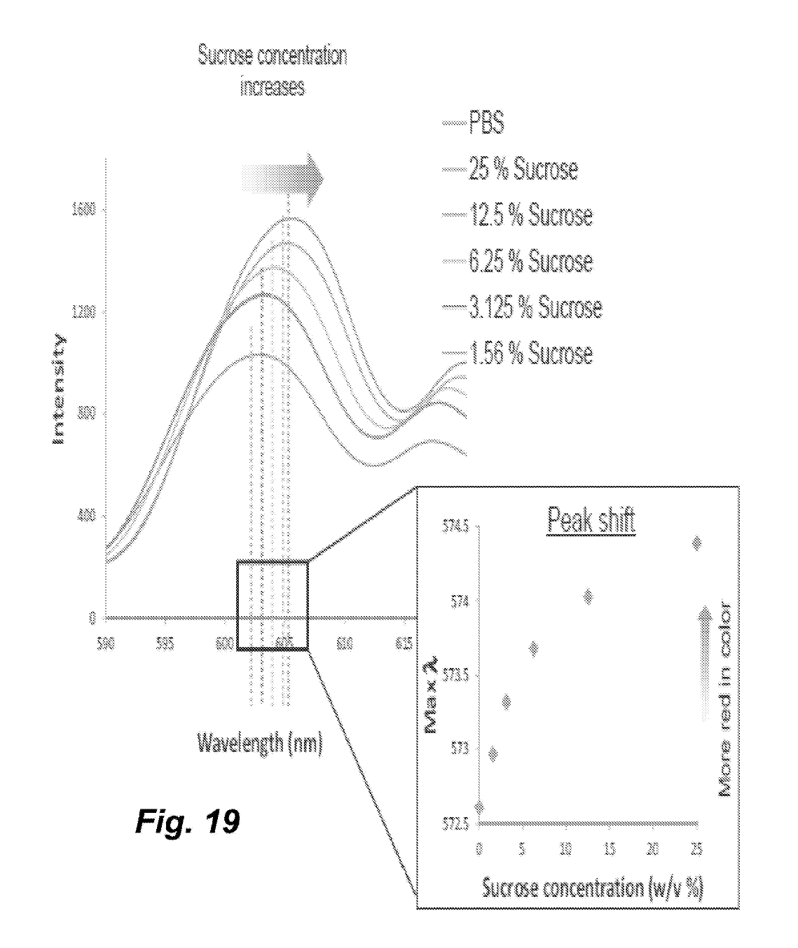

The results are provided in FIG. 19 which shows the measured optical spectra from porous silicon rugate film at different concentrations of sucrose. The result indicates that the sucrose could be detected through the induced fibrotic matrix, with subsequent detection successful even though the porous silicon film was covered with thick fibrotic tissue.

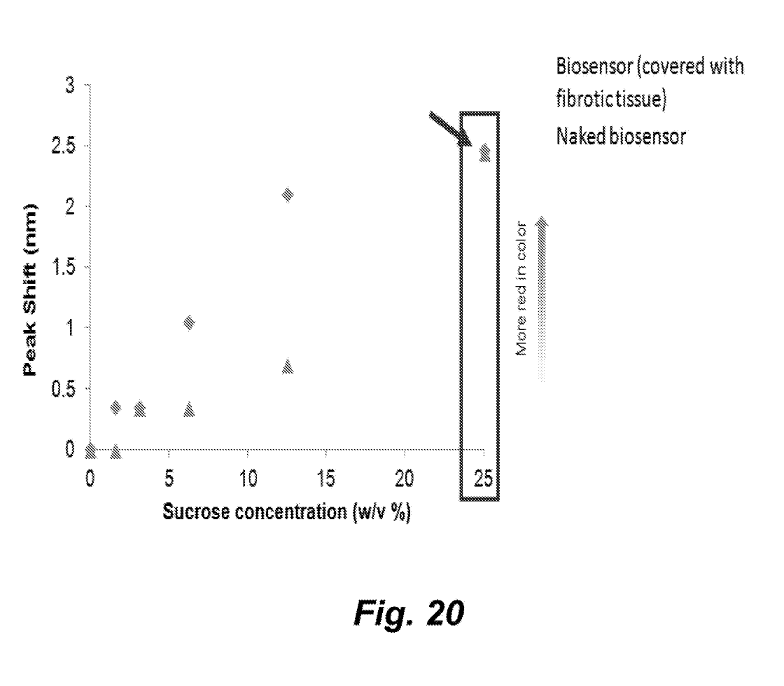

FIG. 20 shows a comparison of porous silicon rugate film response to different sucrose concentrations, with and without induced fibrotic matrix on the porous surface. The measured optical response indicates that the sensor functions normally, even with fibrotic tissue on the surface.

### EXAMPLE 10

### Measurement of Optical Signal from Implanted Porous Silicon Biosensor

The biosensor described herein relies on the measurement of reflected light, and thus if the biosensor is implant for use, there is an issue whether the light signal can be transmit through skin tissue. Accordingly, we tested whether, and for how long (weeks), the optical signal from an implanted biosensor could be measured through skin

The mice were anesthetized under insoflurine. Incision was made on their dorsal right flank. Subcutaneous pocket was opened using a blunt tweezers. The biosensor is then inserted into the pocket. Wound was closed by stitching. At certain time points, the mice were again anesthetized, interferrometric reflectance light probe was aligned perpendicular to the implanted biosensor. The reading was then made and recorded.

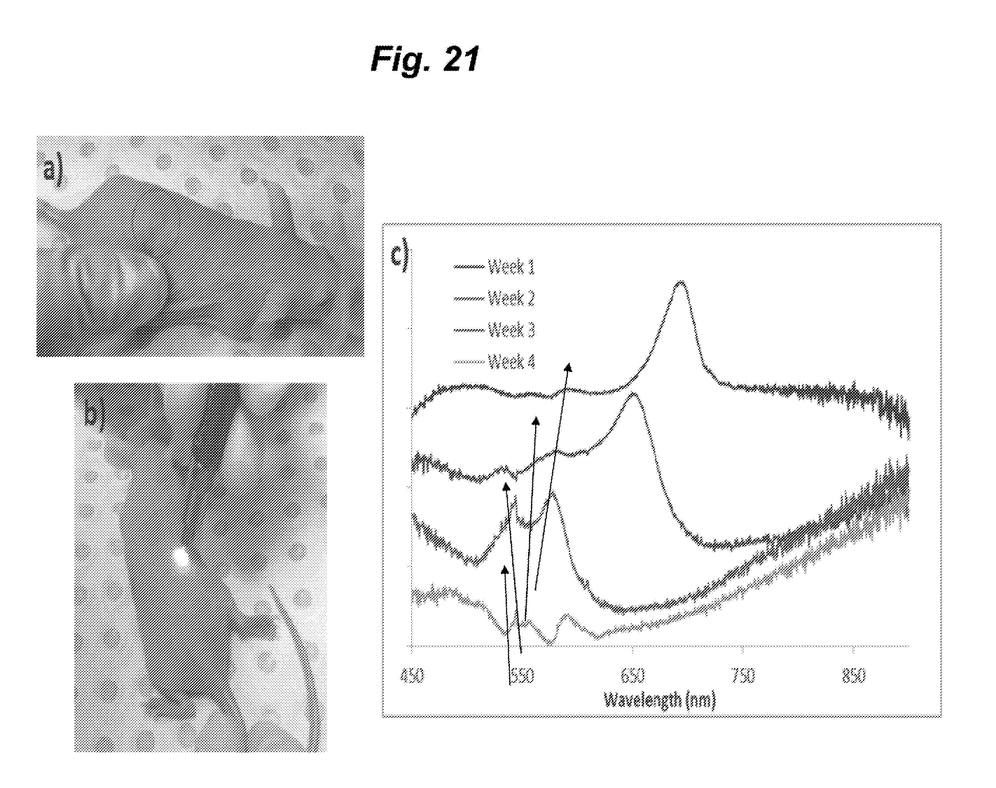

The results are provided in FIG. 21. Panel A shows porous silicon film implanted into hairless mouse. Panel B shows the optical measurement through the skin of the mouse. Panel C shows the optical spectra of porous silicon film, measured each week over 4 weeks post-implantation.

### EXAMPLE 11

### Assessment of Biosensor Function Using Anti-Mouse Albumin Functionalised pSi

As a proof of concept for in vivo biosensing, the pSi biosensor was functionalised with anti-mouse albumin and subcutaneously implanted for a period of 17 days. Over time, serum albumin from the mouse would bind to the antibody within the porous surface and cause a red shift of the optical signal.

The implantation of the biosensor was following the same procedure described previously herein. The biosensor was made using method 1 (thermal hydrocarbonization), with anti-mouse albumin conjugated to the surface.

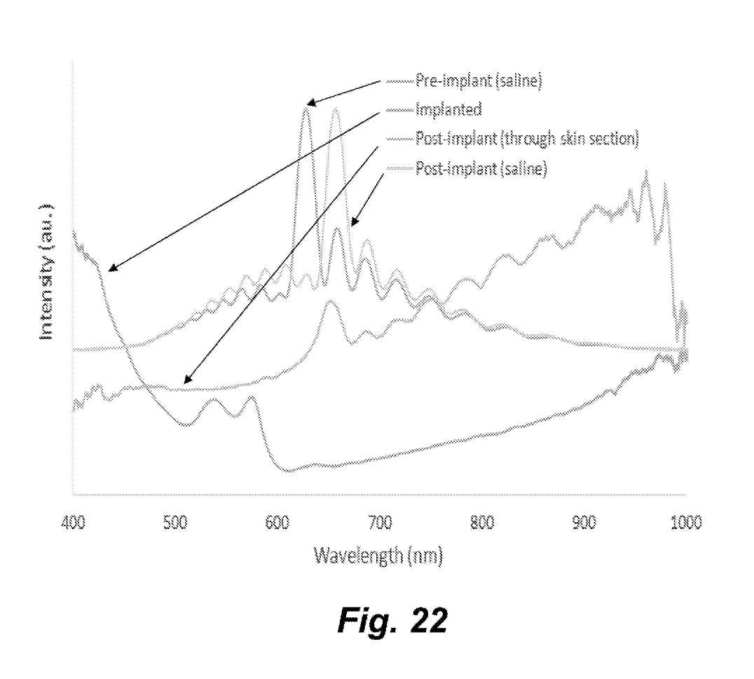

The results are shown in FIG. 22. Reflectance spectra from the anti-mouse albumin functionalised pSi biosensor, pre-implant, implanted (through skin, weak signal), through removed skin section and post implant (biosensor surface removed from skin section). It can be seen that the signal of the post-implant surface has red shifted by approximately 30 nm, indicating protein binding within the pores.

### EXAMPLE 12

### Animal Wellbeing Following Porous Silicon Implant

Apart from functionality, the effect of implantation on the health of the animal was also determined. It is necessary that the implantation causes minimal impact on the animal.

To this end, a biosensor was implanted on the right flank of mice. Weight, as a general health indicator was monitored daily. The results of this analysis demonstrated that mice tolerated the biosensor well, with no health issues nor adverse effects indicated from any change in weight.

### EXAMPLE 13

### Histology of Implant Site: H & E Staining

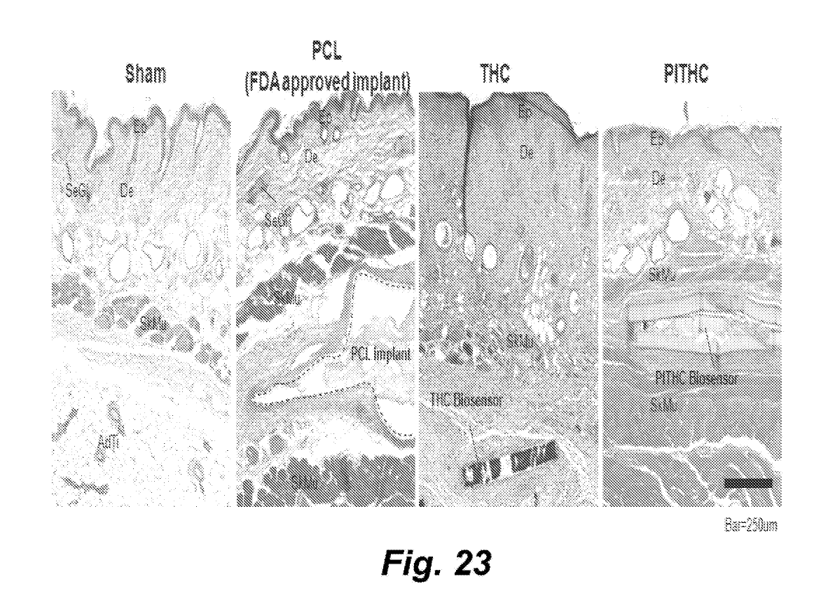

The impact of subcutaneous implantation of the biosensor on tissue in close proximity was investigated. Each mouse received biosensor (PITHC), together with polycaprolactone (PCL) and was implanted on the right flank. Tissue harvested at endpoint and H&E stained. All implants were placed in a subcutaneous area. FIG. 23 shows 1 week post surgery histological sections for sham, positive control and porous silicon biosensor implant sites [De: dermis; SkMu: Skeletal muscle; AdTi: Adipose tissue; SeGl: Sebaceous gland]. [1 week post-surgery].

The results show that PITHC is similar to Sham and PCL, which induced no necrosis. A clear elimination of cytotoxicity effect was seen on THC, where necrosis and epidermis/dermis hypodyplasia occurred. PITHC also induced normal tissue on growth similar to PCL (which is a FDA approved implant). THC showed poor and non-structural on growth. Rugate microstructures were still preserved in PITHC, suggesting that PITHC still possesses rugate optical characteristics.

### EXAMPLE 14

### Histology of Implant Site: Sirius Red Staining

The impact of subcutaneous implantation of the biosensor on tissue in close proximity to the biosensor was investigated. The biosensor was implanted on the right flank of mice, tissue harvested at endpoint and sirius red stained (1 week post-surgery).

The results are shown in FIG. 23. A Col1 capsule was formed around PCL implant, Col1 was not identifiable around the biosensor. The results indicate that the extent of fibrotic tissue formation is even lesser than anticipated. Collagen 3, a collagen subtype which exists during wound healing is visible in PITHC, Sham and PCL. This indicates that PITHC is similar to Sham and PCL, and as such wound healing and tissue formation around implants are normal.

### EXAMPLE 15

### Histology of Implant Site: Staining Against the Inflammatory Regulator IL-6

The inflammatory response of the surgical site and implants was investigated. Histological sections of a sham, PCL, THC biosensor and PlTHC biosensor surface were stained against the inflammatory marker IL-6 and compared.

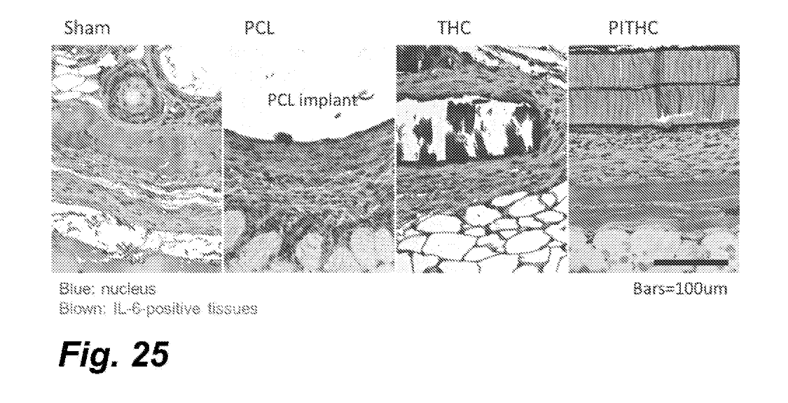

The results are shown in FIG. 25 and indicate that PlTHC is comparable to PCL in terms of inflammatory response. Again, a clear indication of the breakthrough from a conventional THC biosensor, which induced more inflammatory response around the implanted material. This is a positive sign in terms of recovery from surgery and fibrotic tissue formation around the implanted material.

### EXAMPLE 16

### Histology of Distal Tissue: Liver

The impact of subcutaneous implantation on distal tissues and organs was also investigated. Biosensor was implanted on the right flank of mice. Liver was harvested at endpoint (1 week post-surgery) and stained with H&E.

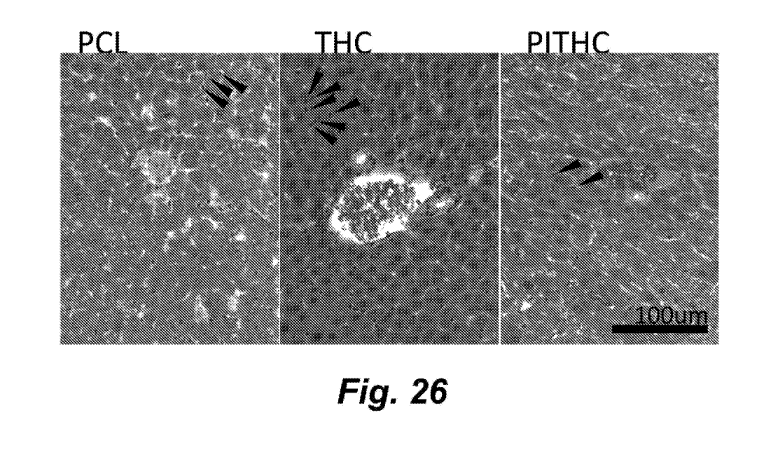

The results are shown in FIG. 26. H & E staining of liver section from mice with PCL and porous silicon biosensor implant respectively. There was no sign of hepatic necrosis and inflammatory cells infiltration in either group. Hepatocyte morphologies were normal for both groups. No pigment deposition was observed in either group, with both resembling the normal histology of liver. The results indicate that there is no toxicity induced by the biosensor.

### EXAMPLE 17

### Histology of Distal Tissue: Spleen

The impact of subcutaneous implantation on distal tissues and organs was also investigated. Biosensor was implanted on the right flank of mice. Spleen was harvested at endpoint (1 week post-surgery) and stained with H&E.

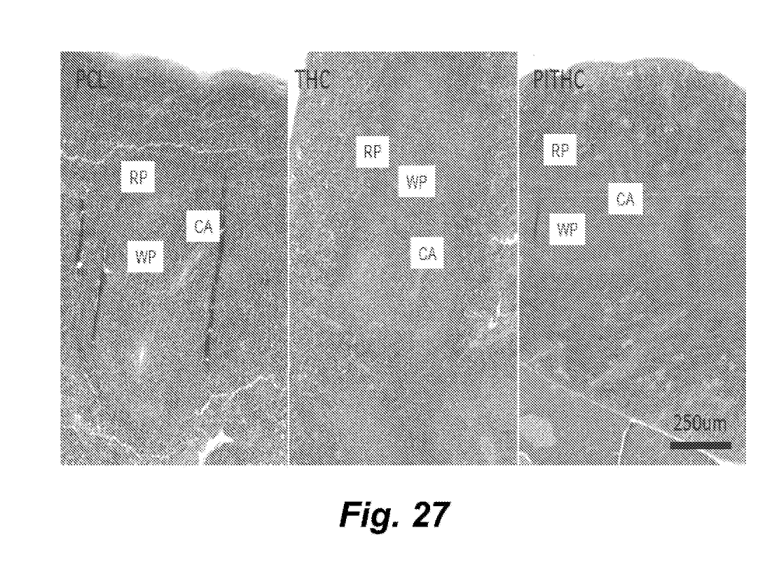

The results are shown in FIG. 27 [RP: Red Pulp; WP: White Pulp; CA: Central Arteriole]. H & E staining of spleen section from mice with PCL and porous silicon biosensor implant respectively. No obvious difference in RP WP size between groups. Magnified images show no difference of cellularity between ctrl and test group in RP, WP. T-cell B-cell population were not significantly different. The result indicates that there was no systemic immunological response caused by the biosensor.

### EXAMPLE 18

### Optical Readings of Porous Silicon Biosensor in the Bovine and Milk/Blood Analysis

(i) Porous Silicon Fabrication

Photonic porous silicon surfaces may be prepared as described in Example 4. The surfaces may be functionalised by thermal hydrocarbonization and functionalised with luteinizing hormone as described in Example 5.

(ii) Insertion of Chip

To evaluate the ability of the biosensor to report hormone levels in vivo it needs to be implanted subcutaneously. To minimise pain and distress to the cow, it will first be immobilised in a cattle crush. Cattle crushes are specifically designed to reduce stress and chance of injury to the animal during examination and minor procedures. Each cow may receive up to 3 implants on up to 6 occasions. The biosensor will be implanted into the base of the ear, escutcheon region of the udder and the caudal tail fold. The cow will be administered with a local anaesthetic (5 ml lignocaine) subcutaneously to form a bleb which numbs the area where the biosensor will be implanted. Once the area has been adequately numbed with the local anaesthetic, betadine will be liberally applied to sterilise the surgical site. A small incision (˜1 cm) will then be made with a scalpel blade. A subcutaneous pocket will be formed using blunt dissection with sterile curved artery forceps and the biosensor will be inserted beneath the skin. After implantation the surgical site will be closed with sutures. The surgical site will be closely monitored daily to ensure healing and to pick up any signs of infection. If any signs of infection, such as redness, swelling or weeping of the wound become evident veterinary attention will be called upon. If required antibiotics (eg. penicillin G) will be administered.

(iii) Milk Collection

Milk will be collected daily throughout the study for analysis of degradation products. The cow will be held in a cattle crush to separate her from her calf for 1 hour each to enable the rapid collection of 100 ml of milk. Analytes (or other biological parameters) can also be measured in the milk using a sensor as described herein, or by other methods. Breakdown products of the sensor can also be measured in the milk.

(iv) Biosensor Reading

The biosensor will be read with a bifurcated fibre optic probe connected to a tungsten light source (incident light) and an Ocean Optics USB2000 spectrometer (reflected light), while the cow is held in a cattle crush.

(v) Blood Collection

The cow will be immobilised in a cattle crush. Blood will be collected via the jugular vein. A 18G needle will be inserted into the vein and 10 ml of blood will be collected at each time point. The study will require the collection of 5 blood samples for each experiment. One blood sample will be collected prior to the implantation of the biosensor. Following the implantation of the biosensor, blood will be collected every 7 days for four weeks (on day 7, 14, 21 and 28). For subsequent experiments blood will be collected every 2-3 days for a 4 week period. There will be at least a four week break between each 4 week blood collection period. Analytes and/or breakdown products can, for example, be measured in the blood.

As used herein, the singular forms “a,” “an,” and “the” may refer to plural articles unless specifically stated otherwise.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

The term “about” or “approximately” means an acceptable error for a particular value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurements method. For removal of doubt, it shall be understood that any range stated herein that does not specifically recite the term “about” before the range or before any value within the stated range inherently includes such term to encompass the approximation within the deviation noted above.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Also, it must be noted that, as used herein, the singular forms “a”, “an” and “the” include plural aspects unless the context already dictates otherwise.

The subject headings used herein are included only for the ease of reference of the reader and should not be used to limit the subject matter found throughout the disclosure or the claims. The subject headings should not be used in construing the scope of the claims or the claim limitations.

Reference to any prior art in this specification is not, and should not be taken as, an acknowledgment or any form of suggestion that this prior art forms part of the common general knowledge in any country.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

Although the present disclosure has been described with reference to particular examples, it will be appreciated by those skilled in the art that the disclosure may be embodied in many other forms.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of detecting a biological parameter in a subject, the method comprising:
implanting a sensor into the subject, wherein the sensor comprises an optical reflectance property between 400 and 1200 nm which is responsive to the biological parameter;
detecting an optical reflectance property between 400 and 1200 nm through the skin of the subject from the implanted sensor, and
using the optical reflectance property to detect the biological parameter.

2. The method according to claim 1, wherein the method comprises detecting an optical reflectance property between 650 nm and 800 nm.

3. The method according to claim 1 or 2, wherein the method comprises detecting an optical reflectance property between 550 and 750 nm.

4. The method according to any one of claims 1 to 3, wherein the optical reflectance property comprises one or more reflectance wavelength spectrum and/or one or more discrete reflectance wavelengths.

5. The method according to any one of claims 1 to 4, wherein the optical reflectance property comprises a change of the photonic peak of the optical reflectance property.

6. The method according to claim 5, wherein the change of the photonic peak comprises an increase in the wavelength of the photonic peak.

7. The method according to claim 5 or 6, wherein the change of the photonic peak is indicative of the value of the biological parameter in the subject.

8. The method according to any one of claims 1 to 7, wherein the sensor comprises one or more porous silicon layers.

9. The method according to claim 8, wherein the sensor comprises a single layer porous silicon layer.

10. The method according to claim 8, wherein the sensor comprises a plurality of porous silicon layers.

11. The method according to claim 10, wherein the plurality of porous silicon layers comprise a Bragg reflector.

12. The method according to claim 10 or 11, wherein the plurality of porous silicon layers comprise a rugate filter.

13. The method according to any one of claims 10 to 12, wherein the sensor comprises microcavities between porous silicon layers.

14. The method according to any one of claims 8 to 13, wherein the one or more porous silicon layers comprise pores of a size of 5 to 500 nm.

15. The method according to any one of claims 8 to 14, wherein the sensor comprises a film comprising the one or more silicon layers and/or particles comprising the one or more silicon layers.

16. The method according to any one of claims 1 to 15, wherein the biological parameter is selected from the presence or absence of an analyte, the concentration of an analyte, the change in concentration of an analyte, temperature, pH, oxygen level, carbon dioxide level, and osmolarity.

17. The method according to any one of claims 1 to 16, wherein the biological parameter is the concentration of an analyte.

18. The method according to claim 16 or 17, wherein the analyte comprises one or more of a hormone, a growth factor, an antibody, an enzyme, a drug, a protein, a ligand, a nucleic acid, a small molecule, a metabolite, a cofactor, an amino acid, a vitamin, a lipid, a carbohydrate, a sugar, a cell and/or a component thereof, an inflammatory marker, a toxin, a pesticide, a metal ion, a pathogen, a bacteria, a virus, an antigen, insulin, and an antibiotic.

19. The method according to any one of claims 16 to 18, wherein the analyte comprises a hormone whose level is indicative of pregnancy.

20. The method according to any one of claims 16 to 18, wherein the analyte comprises a hormone whose level is indicative of ovulation status.

21. The method according to claim 20, wherein the hormone comprises luteinizing hormone.

22. The method according to any one of claims 16 to 21, wherein the sensor comprises an analyte binding molecule and the optical reflectance property changes upon binding of the analyte to the analyte binding molecule.

23. The method according to claim 22, wherein the analyte binding molecule comprises an antibody and/or an antigen binding fragment thereof.

24. The method according to claim 23, wherein the antibody and/or an antigen binding fragment thereof is attached to the sensor through a carbohydrate group on the antibody and/or antigen binding fragment thereof.

25. The method according to claim 23 or 24, wherein the analyte comprises luteinizing hormone and the analyte binding molecule comprises an antibody to luteinizing hormone and/or an antigen binding fragment thereof.

26. The method according to any one of claims 1 to 25, wherein the sensor is implanted under the skin of the subject.

27. The method according to any one of claims 1 to 26, wherein the subject is a human subject, a bovine animal, an ovine animal, a porcine animal, an equine animal, or a caprine animal.

28. The method according to any one of claims 1 to 27, wherein the sensor further comprises an optical reflectance property that is indicative of the identity of the subject.

29. The method according to any one of claims 8 to 28, wherein the one or more porous silicon layers are pre-treated to improve biocompatibility.

30. The method according to any one of claims 1 to 31, wherein the method is used to determine whether the subject is ovulating, to determine whether the subject is pregnant, to determine the health of the subject, to determine whether the subject is suffering from or susceptible to a disease, condition or state, to determine whether a subject is in need of treatment, and to determine the health and/or fitness of the subject.

31. A method of determining the ovulation status of a subject, the method comprising:
implanting into the subject a sensor comprising one or more porous silicon layers and a luteinizing hormone binding molecule, wherein the sensor comprise an optical reflectance property between 400 and 1200 nm which is responsive to binding of leutinizing hormone to the sensor;
detecting an optical reflectance property between 400 and 1200 nm through the skin from the implanted sensor; and
determining the ovulation status of the subject on the basis of the optical reflectance property detected.

32. A method of determining the pregnancy status of a subject, the method comprising:
implanting into the skin a sensor comprising one or more porous silicon layers and a molecule that binds to an analyte indicative of pregnancy status, wherein the sensor comprise an optical reflectance property between 400 and 1200 nm which is responsive to binding of the analyte to the sensor;
detecting an optical reflectance property between 400 and 1200 nm through the skin from the implanted sensor; and
determining the pregnancy status of the subject on the basis of the optical reflectance property detected.

33. A sensor for detecting a biological parameter, the sensor comprising one or more porous silicon layers, wherein the sensor comprises an optical reflectance property between 400 and 1200 nm that is responsive to the biological parameter.

34. The sensor according to claim 33, wherein the sensor comprises an optical reflectance property between 650 nm and 800 nm.

34. The sensor according to claim 33 or 35, wherein the sensor comprises an optical reflectance property between 550 and 750 nm.

35. The sensor according to any one of claims 33 to 34, wherein the optical reflectance property comprises a photonic peak of the optical reflectance property.

36. The sensor according to any one of claim 33 or 34, wherein the biological parameter is selected from the presence or absence of an analyte, the concentration of an analyte, the change in concentration of an analyte, temperature, pH, oxygen level, carbon dioxide level, and osmolarity.

37. The sensor according to claim 36, wherein the analyte comprises one or more of a hormone, a growth factor, an antibody, an enzyme, a drug, a protein, a ligand, a nucleic acid, a small molecule, a metabolite, a cofactor, an amino acid, a vitamin, a lipid, a carbohydrate, a sugar, a cell and/or a component thereof, an inflammatory marker, a toxin, a pesticide, a metal ion, a pathogen, a bacteria, a virus, an antigen, insulin, and an antibiotic.

38. The sensor according to claim 36 or 37, wherein the sensor comprises an analyte binding molecule and the optical reflectance property changes upon binding of the analyte to the analyte binding molecule.

39. The sensor according to claim 38, wherein the analyte binding molecule is covalently linked to the one or more porous silicon layers.

40. The sensor according to claim 38 or 39, wherein the analyte binding molecule comprises one or more of an antibody and/or an antigen binding fragment thereof.

41. The method according to claim 40, wherein the antibody and/or an antigen binding fragment thereof is attached to the sensor through a carbohydrate group on the antibody and/or antigen binding fragment thereof.

42. The sensor according to any one of claims 37 to 41, wherein the analyte comprises a hormone whose level is indicative of pregnancy.

43. The sensor according to claims any one of claims 37 to 41, wherein the analyte comprises a hormone whose level is indicative of ovulation status.

44. The sensor according to claim 43, wherein the hormone comprises luteinizing hormone.

45. The sensor according to any one of claim 37 to 41 or 43, wherein the analyte comprises luteinizing hormone and the analyte binding molecule comprises an antibody to luteinizing hormone and/or an antigen binding fragment thereof.

46. The sensor according to any one of claims 33 to 45, wherein the sensor comprises a single layer porous silicon layer.

47. The sensor according to any one of claims 33 to 45, wherein the sensor comprises a plurality of porous silicon layers.

48. The sensor according to claim 47, wherein the plurality of porous silicon layers comprise a Bragg reflector.

49. The sensor according to claim 47 or 48, wherein the plurality of porous silicon layers comprise a rugate filter.

50. The sensor according to any one of claims 45 to 47, wherein the sensor comprises microcavities between porous silicon layers.

51. The sensor according to any one of claims 39 to 50, wherein the one or more porous silicon layers comprise pores of a size of 5 to 500 nm.

52. The sensor according to any one of claims 33 to 51, wherein the sensor comprises a film comprising the one or more silicon layers and/or particles comprising the one or more silicon layers.

53. The sensor according to any one of claims 33 to 52, wherein the one or more silicon layers are pre-treated to improve biocompatibility.

54. A method of detecting a biological parameter, the method comprising using a sensor according to any one of claims 33 to 53 to detect the biological parameter.

55. The method according to claim 54, wherein the biological parameter is a biological parameter in vitro.

56. The method according to claim 54, wherein the biological parameter is a biological parameter in vivo.

57. The method according to claim 37 to 42 or 56, wherein the sensor is implanted under the skin of a subject and the optical reflectance property of the implanted sensor is detected through the skin of the subject.

58. A method of determining the ovulation status of a subject, the method comprising implanting a sensor according any one of claims 43 to 53 under the skin of a subject and using the implanted sensor to determine the ovulation status of the subject.

59. A method of determining the pregnancy status of a subject, the method comprising implanting a sensor according any one of claims 42 or 46 to 53 under the skin of a subject and using the implanted sensor to determine the pregnancy status of the subject.

60. A method of identifying a subject, the method comprising using a sensor according to any one of claims 33 to 53 to tag the subject and thereby identify the subject.
